(19) United States
(12) Patent Application Publication
(10) Pub. No.: US 2015/0284472 A1
(43) Pub. Date: Oct. 8, 2015

(54) COMPOSITIONS AND METHODS FOR TREATING PROTEINOPATHIES

(71) Applicant: GENZYME CORPORATION, Cambridge, MA (US)

(72) Inventors: Sergio Pablo Sardi, Newton, MA (US); Lamya Shihabuddin, West Newton, MA (US); Seng Cheng, Natick, MA (US)

(21) Appl. No.: 14/440,818

(22) PCT Filed: Nov. 4, 2013
(86) PCT No.: PCT/US2013/068242

§ 371 Date: May 5, 2015

Related U.S. Application Data
(60) Provisional application No. 61/722,434, filed on Nov. 5, 2012.

(51) Int. Cl. C07K 16/40; A61K 38/47; A61K 35/76; A61K 9/00

(52) U.S. Cl. CPC C07K 16/40; A61K 9/0019; A61K 38/47; A61K 35/76; C12Y 302/01045

(57) ABSTRACT

This disclosure relates to methods for improving neural function in a mammal with a proteinopathy comprising administering a therapeutically effective amount of an agent that increases glucocerebrosidase activity in the mammal. Also disclosed are methods for reducing toxic lipids, reducing α-synuclein, and/or inhibiting the accumulation of protein aggregates in a mammal with a proteinopathy comprising administering a therapeutically effective amount of an agent that increases glucocerebrosidase activity.

### RELATED APPLICATIONS

This application claims the benefit of priority of U.S. Provisional Patent Application No. 61/722,434, filed Nov. 5, 2012, which is hereby incorporated by reference in its entirety.

### DESCRIPTION OF THE INVENTION

In medicine, proteinopathy refers to a class of diseases in which certain proteins become structurally abnormal, and thereby disrupt the function of cells, tissues and organs of the body. Often the proteins fail to fold into their normal configuration. In this misfolded state, the proteins can become toxic in some way (a gain of toxic function) or they can lose their normal function. The proteinopathies include diseases such diseases as Alzheimer's disease, Parkinson's disease, prion disease, type 2 diabetes, amyloidosis, and a wide range of other disorders.

Proteinopathies are widespread throughout the population. For example, nearly one million people in the US are living with Parkinson's disease and as many as 5.1 million Americans have Alzheimer's disease. There are currently no cures for these diseases, and many of the molecular mechanisms underlying the disease and progression of the disease are unknown.

Although there are no cures for these devastating diseases, it is believed that certain symptoms may be alleviated. There is a need in the art to develop therapeutics effective in alleviating or managing the symptoms associated with proteinopathies.

### SUMMARY

This disclosure relates to methods and compositions for treating proteinopathies. One aspect relates to a method for improving neural function in a mammal with a proteinopathy comprising administering a therapeutically effective amount of an agent that increases glucocerebrosidase activity in the mammal. A proteinopathy refers to a disease (e.g., a neurodegenerative disease) caused by a malformed protein and/or accumulation of proteins. This class of diseases is characterized by structurally abnormal proteins that can become toxic, lose their normal function, and/or disrupt the function of cells. A further aspect relates to a method for improving neural function in a mammal suffering from or at risk of suffering from a proteinopathy comprising administering a therapeutically effective amount of an agent that increases glucocerebrosidase activity in the mammal.

Proteinopathies, when present in the central nervous system, can result in an impairment of cognitive function. Cognitive function or neural function refers to memory capabilities, attention, language, decision making, problem solving, and the like. In aspects, the methods of the invention relate to improving cognitive function, e.g., memory function, by increasing glucocerebrosidase activity in the mammal.

Another aspect relates to a method for reducing toxic lipids (e.g., glucosylsphingosine), reducing α-synuclein, reducing tau, or inhibiting/reducing the accumulation of protein aggregates in a mammal with a proteinopathy comprising administering a therapeutically effective amount of an agent that increases glucocerebrosidase activity. A further aspect relates to a method for reducing toxic lipids (e.g., glucosylsphingosine), reducing α-synuclein, reducing tau, or inhibiting the accumulation of protein aggregates in a mammal suffering from or at risk of suffering from a tauopathy comprising administering a therapeutically effective amount of an agent that increases glucocerebrosidase activity. The increase of glucocerebrosidase activity in a mammal can lead to beneficial histological changes. Notably, increasing glucocerebrosidase activity has been shown to reduce toxic protein species in subjects with proteinopathies. Toxic lipids such as glucosylsphingosine accumulate in the CNS and can act as a neurotoxin. α-synuclein is a protein encoded by the SNCA gene in humans. α-synuclein can aggregate to form insoluble fibrils in pathological disorders characterized by Lewy bodies. These disorders, known as synucleinopathies, include, for example, Parkinson's disease and Lew Body dementia. Increasing glucocerebrosidase activity can also reduce other protein aggregates in cells, such as, for example, tau and ubiquitin. In each case, the abnormal forms of the protein are contributing, at least in part, to the disease state of the mammal.

Other aspects of the disclosure relate to a method for reducing glucocerebroside lipid levels in a mammal with a proteinopathy comprising administering a therapeutically effective amount of a small molecule inhibitor of glucocerebroside synthase or a positive modulator of glucocerebrosidase. A further aspect relates to a method for reducing glucocerebroside lipid levels in a mammal suffering from or at risk of suffering from a tauopathy comprising administering a therapeutically effective amount of a small molecule inhibitor of glucocerebroside synthase or a positive modulator of glucocerebrosidase. Substrate reduction therapy has been described previously (see, for example, McEachern K A et al. (2007) Mol. Genet. Metab. 91:259-67; Cabrera-Salazar M A et al. (2012) PLoS One 7:e43310; and U.S. Pat. No. 8,168,587, each of which are incorporated by reference in their entirety). Also disclosed are methods for improving neural function in a mammal in need thereof (e.g., a mammal suffering from or at risk of suffering from a proteinopathy) and methods for preventing, inhibiting, or reducing loss of neural function in a mammal in need thereof comprising administering a therapeutically effective amount of a small molecule inhibitor of glucocerebroside synthase or a positive modulator of glucocerebrosidase to the mammal.

A further aspect relates to a method for preventing loss of neural function in a mammal in need thereof comprising administering a therapeutically effective amount of an agent that increases glucocerebrosidase activity. In one embodiment, the mammal is suffering from or at risk of suffering from a tauopathy. Augmenting glucocerebrosidase activity in a mammal suffering from or at risk of suffering from a proteinopathy may prevent the cognitive impairment, e.g., memory loss and decline in neural function, associated with the disease. In embodiments, this method is beneficial for subjects who may have been diagnosed with a proteinopathy but are not yet experiencing the typical signs of cognitive impairment associated with the disease state. Also, those who are at risk due to, for example, a mutation in the subject or the subject's family lineage known to cause a proteinopathy may also benefit from this therapeutic method.

In some of the above embodiments, the mammal is a human.

In some of the above embodiments, the mammal has been diagnosed with a disease selected from the group consisting of Alzheimer's disease, Gaucher disease, frontotemporal dementia, progressive supranuclear palsy, Parkinsonism, Parkinson's disease, Lytico-Bodig disease, dementia with Lewy bodies, tangle-predominant dementia, dementia pugilistica, Pick's disease, corticobasal degeneration, Argyrophilic grain disease, ganglioglioma and gangliocytoma, meningioangiomatosis, subacute sclerosing panencephalitis, lead encephalopathy, tuberous sclerosis, Hallervorden-Spatz disease, and lipofuscinosis.

In some of the above embodiments, the mammal may have reduced glucocerebrosidase activity prior to administration of the agent.

In some of the above embodiments, the mammal may have a one or more mutations in the glucocerebrosidase 1 (GBA1) gene. GBA1 mutations are well known in the art, and nonlimiting examples are described herein (e.g., D409V mutation).

In some of the above embodiments, the method reduces tau, α-synuclein, and/or toxic lipids (e.g., glucosylsphingosine). In related embodiments, toxic glucosylsphingosine is reduced by at least 10%, 20%, 30%, 40%, 50%, 60%, 70%, 80%, 90%, or more. In related embodiments, toxic glucosylsphingosine is reduced to a level not significantly different than a mammal without a proteinopathy.

In some of the above embodiments, the proteinopathy is associated with protein aggregates (e.g., ubiquitin, tau, and/or α-synuclein). In related embodiments, the method involves inhibiting the accumulation of protein aggregates (e.g., protein aggregates comprising ubiquitin, tau, and/or α-synuclein). In some embodiments, the proteinopathy is a tauopathy (e.g., Alzheimer's disease, frontotemporal dementia, progressive supranuclear palsy, Parkinsonism, Parkinson's disease, Lytico-Bodig disease, dementia with Lewy bodies, tangle-predominant dementia, dementia pugilistica, Pick's disease, corticobasal degeneration, Argyrophilic grain disease, ganglioglioma and gangliocytoma, meningioangiomatosis, subacute sclerosing panencephalitis, lead encephalopathy, tuberous sclerosis, Hallervorden-Spatz disease, and lipofuscinosis). In some embodiments, the proteinopathy is a synucleinopathy.

In some of the above embodiments, the agent is (or contains) a small molecule, an antibody, a nucleic acid molecule, or a polypeptide. In embodiments, the agent is a nucleic acid encoding a GBA1 gene or equivalent thereof (e.g., fragment, analog, or derivative thereof that encodes a polypeptide that catalyzes the cleavage of glucocerebroside). In other embodiments, the agent is a GBA1 polypeptide or equivalent thereof (e.g., fragment, analog, or derivative thereof that catalyzes the cleavage of glucocerebroside). In embodiments, the agent is an antibody or fragment thereof that specifically binds to GBA1. In embodiments, the agent is a small molecule (e.g., small molecule activator). In embodiments, the agent is a chaperone. In embodiments, the methods involve administering a second agent that is beneficial in treating a symptom associated with a proteinopathy, a synucleinopathy, a tauopathy, or the like.

In some of the above embodiments, the agent is a virus/viral vector. In embodiments, the virus comprises a nucleic acid encoding a GBA1 gene or an equivalent thereof. In related embodiments, the GBA1 gene or equivalent thereof is operably linked to a promoter that regulates expression of the GBA1 protein (e.g., promoter is capable of expressing the GBA1 gene or equivalent thereof in neurons of the central nervous system, including but not limited to, a human β-glucuronidase promoter or a cytomegalovirus enhancer linked to a chicken β-actin promoter).

In some of the above embodiments, the agent is an adeno-associated virus (AAV). In embodiments, the AAV comprises an AAV1, AAV2, AAV3, AAV4, AAV5, AAV6, AAV7, AAV8, AAVrh8, AAV9, AAV10, AAVrh10, AAV11, or AAV12 serotype capsid. In embodiments, the AAV comprises an AAV serotype capsid from Clades A-F. In embodiments, the AAV comprises an AAV1, AAV2, AAV3, AAV4, AAV5, AAV6, AAV7, AAV8, AAVrh8, AAV9, AAV10, AAVrh10, AAV11, or AAV12 inverted terminal repeat (ITR). In embodiments, the AAV comprises an AAV ITR from Clades A-F.

In some embodiments, the ITR and the capsid are derived from the same AAV serotype. In other embodiments, the ITR and the capsid are derived from different AAV serotypes.

In some embodiments, the AAV is a self-complementary AAV.

In one embodiment, the nucleic acid comprises a first heterologous polynucleotide sequence encoding a GBA1 transgene and a second heterologous polynucleotide sequence encoding a complement of the GBA1 transgene, wherein the first heterologous polynucleotide sequence can form intrastrand base pairs with the second polynucleotide sequence. In related embodiments, the first heterologous polynucleotide sequence and the second heterologous polynucleotide sequence are linked by a mutated AAV ITR (e.g., the mutated AAV ITR comprises a deletion of the D region and comprises a mutation of the terminal resolution sequence).

In some of the above embodiments, the agent is in a pharmaceutical composition. In related embodiments, the pharmaceutical composition further comprises a pharmaceutically acceptable carrier.

In some of the above embodiments, the agent or pharmaceutical composition is administered via an oral route, via an intravascular route, via an intravenous route, via an intramuscular route, by direct absorption through mucous membrane tissues (e.g., nasal, mouth, vaginal, rectal, and the like), via a transdermal route, via an intradermal route, via the central nervous system (CNS), via the spinal cord, via an intracranial route, via an intraventricular route, via an intrathecal route, or via an intracerebral route.

In some of the above embodiments, the agent or pharmaceutical composition is administered by injection. In embodiments, the agent or pharmaceutical composition is administered into the CNS (e.g., via direct injection into the spinal cord, via intrathecal injection, via intracerebroventricular injection, or via intrahippocampal injection).

In some of the above embodiments, the method comprises increasing the glucocerebrosidase activity over baseline levels in the neuron (e.g., by at least 1.5 fold, 2 fold, 3 fold, 4 fold, 5 fold, 6 fold, 7 fold, 8 fold, 9 fold, 10 fold, or more over baseline levels in the neuron).

Additional objects and advantages of the invention will be set forth in part in the description which follows, and in part will be obvious from the description, or may be learned by practice of the invention. The objects and advantages of the invention will be realized and attained by means of the elements and combinations disclosed herein, including those pointed out in the appended claims. It is to be understood that both the foregoing general description and the following detailed description are exemplary and explanatory only and are not restrictive of the invention as claimed. The accompanying drawings, which are incorporated in and constitute a part of this specification, illustrate several embodiments of the invention and, together with the description, serve to explain the principles of the invention.

### BRIEF DESCRIPTION OF THE DRAWINGS

FIGS. 1A-C show the progressive accumulation of tau aggregates in the brains of Gba1^{D409V/D409V} mice. (A) Images show immunostaining with an anti-tau serum (green) and nuclear staining (DAPI, blue) in the hippocampi of 2-, 6- and 12-month-old Gba1^{D409V/D409V} and age-matched wild-type (WT) mice (scale bar, 500 μm). (B) Quantification of Tau-5 immunoreactivity in WT and Gba1^{D409V/D409V} hippocampi at 2, 6, and 12 months shows progressive accumulation of aggregates with age (n>5 per group). (C) Shown are representative immunoblots of hippocampal lysates from 18-month-old Gba1^{D409V/D409V} mice and age-matched controls for AT8, AT180, AT270, Tau-5 and β-tubulin. Each lane represents an independent mouse brain. Clone AT8 antibody shows increased tau phosphorylation (S202/T205) in aged Gba1^{D409V/D409V} mice. No differences between mutant and wild-type mice were observed in total tau levels (Tau-5) or other phosphorylated species (AT180 or AT270). The results are represented as the means±SEM. Bars marked with different letters are significantly different from each other (p<0.05).

FIGS. 2A-F show that CNS administration of AAV-GBA1 reduces glucosylsphingosine levels and reverses memory deficits. Four- and 12-month-old Gba1^{D409V/D409V} mice were given bilateral hippocampal injections of either AAV-EV or AAV-GBA1. Uninjected Gba1^{D409V/D409V} littermates were euthanized at the time of surgeries as baselines for biochemical and histological endpoints (n=8). Age-matched, uninjected wild-type (WT, n=9) mice were used as a positive control. In both cohorts, tissues were collected for biochemical and pathological analysis at 6 months post-injection. (A) Hippocampal expression of the recombinant enzyme 6 months after stereotaxic injections. Image shows glucocerebrosidase immunoreactivity (red) and nuclear (DAPI, blue) stains in an AAV-GBA1-injected Gba1^{D409V/D409V} mouse (scale bar, 400 μm). Inset depicts glucocerebrosidase and nuclear staining in an AAV-EV-injected mouse. Hippocampal administration of AAV-GBA1 into Gba1^{D409V/D409V} mice increased glucocerebrosidase activity (B, red bar, n=11, p<0.05) and promoted clearance of glucosylsphingosine (GlcSph) to WT levels (C; red bar, n=11, p<0.05), whereas AAV-EV treated Gba1^{D409V/D409V} mice showed no change in glucocerebrosidase activity (B, blue bar, n=12, p>0.05) and continued to accumulate GluSph compared to baseline levels (C, black bar, n=8, p<0.05). (D) Pre-surgical evaluation of 4-month-old wild-type (WT) and Gba1^{D409V/D409V} mice revealed no object preference when exposed to two identical objects. The results from trial 1 (training) are shown as white (WT) and purple (Gba1^{D409V/D409V} mice) solid bars. After a 24 h retention period, mice were presented with a novel object. In trial 2 (testing, hatched bars), WT mice investigated the novel object significantly more frequently (n=9, p<0.05). In contrast, Gba1^{D409V/D409V} mice (n=11, blue hatched bar) showed no preference for the novel object, indicating a cognitive impairment. (E) At 2 months post-injection, mice were subjected to the novel object recognition (NOR) test. AAV-GBA1-treated Gba1^{D409V/D409V} mice (n=10, blue hatched bar), but not AAV-EV-treated animals (n=9, red hatched bar), exhibited a reversal of their memory deficit when presented with the novel object during the testing trial. (F) A separate cohort of 12-month-old Gba1^{D409V/D409V} mice were injected with AAV-EV (n=12) or AAV-GBA1 (n=12). Similar to the 4-month-old cohort, reversal of the memory dysfunction was observed when these animals were tested at 2 months post-injection (14 months of age). The results are represented as the means±the SEM. (D-F) The horizontal line demarcates 50% target investigations, which represents no preference for either object (*, significantly different from 50%, p<0.05); (B, C). Bars with different letters are significantly different from each other (p<0.05).

FIGS. 3A-C show that expression of glucocerebrosidase in symptomatic Gba1^{D409V/D409V} mouse hippocampi slows accumulation of aggregated α-synuclein and tau. Two cohorts of Gba1^{D409V/D409V} mice were injected with either AAV-EV or AAV-GBA1 bilaterally into the hippocampus at 4 or 12 months of age. Age-matched, uninjected WT mice were left untreated as positive controls. Gba1^{D409V/D409V} littermates were harvested at the time of the injections as a baseline group. Injected animals were sacrificed 6 months after surgery. Graphs represent hippocampal quantifications of ubiquitin (A), proteinase K-resistant α-synuclein (B) and tau immunoreactivity (C) for the cohorts injected at 4 (left) or 12 (right) months of age. Glucocerebrosidase augmentation in the CNS of symptomatic Gba1^{D409V/D409V} mice reduced the levels of aggregated proteins, although this treatment was less effective in older animals. Images show ubiquitin (A, green), proteinase K-resistant α-synuclein (B, red) and tau (C, green) immunoreactivity in the hippocampi of 18-month-old Gba1^{D409V/D409V} mice treated with AAV-EV or AAV-GBA1. DAPI nuclear staining is shown in blue (scale bar, 100 μm). The results are represented as the means±the SEM with n≧8 per group. Bars with different letters are significantly different from each other (p<0.05).

FIGS. 4A-C demonstrate that glucocerebrosidase augmentation in A53T α-synuclein mouse brain decreases α-synuclein levels. A53T α-synuclein transgenic mice exhibit decreased brain glucocerebrosidase activity. (A) The activity of various lysosomal enzymes was determined in cortical homogenates from homozygous (n=9) and heterozygous (n=8) α-synuclein transgenic and wild-type littermates (n=9). Glucocerebrosidase activity was inversely correlated with α-synuclein levels, with homozygous mice showing a greater reduction of hydrolase activity. The enzymatic activities of two other lysosomal hydrolases, hexosaminidase and β-galactosidase, remained unchanged by the expression of A53T-α-synuclein. (B) Four-month-old A53T α-synuclein mice were each injected with either AAV-GFP (n=6) or AAV-GBA1 (n=5) unilaterally into the right striatum. The left striatum was used as an uninjected control for each animal to reduce the variability in α-synuclein levels between subjects. Four months later, mice were euthanized, and both striata were collected. Robust glucocerebrosidase activity was observed in the AAV-GBA1-injected striata (7-fold over the uninjected contralateral side). Expression of glucocerebrosidase promoted decreased α-synuclein levels in the cytosolic fraction (Tris-soluble, non-membrane-associated; p<0.05). (C) Newborn (P0) A53T-α-synuclein mice were injected with either AAV-GFP or AAV-GBA1 into the lumbar spinal cord. As expected, robust glucocerebrosidase activity was noted in AAV-GBA1-injected mice (3-fold over controls). As in the striatum, expression of glucocerebrosidase reduced α-synuclein levels in the cytosolic fraction (Tris-soluble, non-membrane associated; n=7 per group, p<0.05). Data are represented as the means±the SEM. * denotes statistical significance at p<0.05.

FIGS. 5A-B show that decreased glucocerebrosidase activity leads to α-synuclein accumulation. (A) Depicted are immunohistochemical images showing hippocampal α-synuclein and ubiquitin aggregates in Gba1^{D409V/D409V} Gaucher mice. (B) The percentage of α-synuclein immunoreactivity is quantified for the WT and Gba1^{D409V/D409V} mice. PK=Proteinase K.

FIGS. 6A-D show a characterization of the Gba1^{D409V/D409V} Gaucher mice model synucleinopathies. These mice demonstrated progressive accumulation of ubiquitin (A) and α-synuclein aggregates (B) and glucosylsphingosine accumulation (D). Additionally, these mice demonstrated the memory deficit in the novel object recognition test (C).

FIG. 7 demonstrates that GBA1 augmentation ameliorates Gba1^{D409V/D409V} mouse pathology (preventive study). A novel object recognition test shows that AAV-mediated delivery of glucocerebrosidase into hippocampus of pre-symptomatic (2 month old Gba1^{D409V/D409V}) mouse corrects memory deficit.

FIG. 8 shows that expression of glucocerebrosidase in A53T α-synuclein mouse brain decreases accumulation of Tau aggregates. A53T-α-synuclein transgenic mice were bilaterally injected into the lateral ventricles with either AAV-control or AAV-GBA1 at P0. Age-matched, uninjected WT mice were left untreated as negative controls. Images show immunostaining with an anti-tau serum (green) and nuclear staining (DAPI, blue) in the hippocampi of wild-type (WT) and A53T-α-synuclein overexpressing mice (scale bar, 500 μm).

FIGS. 9A-B show that augmenting glucocerebrosidase activity in the CNS of tau transgenic mice prevents memory dysfunction. (A) Two month-old Tau transgenics were given bilateral hippocampal injections of either AAV-EV or AAV-GBA1. Age-matched, uninjected wild-type (WT; n=8) mice were used as a positive control for the test. The results from trial 1 (training) are shown as white (WT), green (TAU+AAV-EV) or red (TAU+AAV-GBA1) filled bars. After a 24-h retention period, mice were presented with a novel object. In trial 2 (testing, hatched bars), WT mice investigated the novel object significantly more frequently both at 4 or 8 months of age. In contrast, Thy1-TAU22 transgenic mice injected with control virus (n=9; green hatched bar) showed no preference for the novel object, indicating a cognitive impairment at both time points assayed. AAV-GBA1-treated Thy1-TAU22 mice (n=13; red hatched bar) exhibited a trend to memory function improvement 2 months after treatment that was significant when tested at 6 months post-treatment. The results are represented as means±SEM. The horizontal line demarcates 50% target investigations, which represents no preference for either object (*, significantly different from 50%, P<0.05).

### DETAILED DESCRIPTION

### Definitions

Unless defined otherwise, all technical and scientific terms used herein have the same meanings as commonly understood by one of ordinary skill in the art to which this disclosure belongs. Although any methods and materials similar or equivalent to those described herein can be used in the practice or testing of the present invention, exemplary methods, devices, and materials are now described. All technical and patent publications cited herein are incorporated herein by reference in their entirety. Nothing herein is to be construed as an admission that the invention is not entitled to antedate such disclosure by virtue of prior invention.

The practice of the present disclosure will employ, unless otherwise indicated, conventional techniques of tissue culture, immunology, molecular biology, microbiology, cell biology and recombinant DNA, which are within the skill of the art. See, e.g., Michael R. Green and Joseph Sambrook, Molecular Cloning (4^{th} ed., Cold Spring Harbor Laboratory Press 2012); the series Ausubel et al. eds. (2007) Current Protocols in Molecular Biology; the series Methods in Enzymology (Academic Press, Inc., N.Y.); MacPherson et al. (1991) PCR 1: A Practical Approach (IRL Press at Oxford University Press); MacPherson et al. (1995) PCR 2: A Practical Approach; Harlow and Lane eds. (1999) Antibodies, A Laboratory Manual; Freshney (2005) Culture of Animal Cells: A Manual of Basic Technique, 5^{th} edition; Gait ed. (1984) Oligonucleotide Synthesis; U.S. Pat. No. 4,683,195; Hames and Higgins eds. (1984) Nucleic Acid Hybridization; Anderson (1999) Nucleic Acid Hybridization; Hames and Higgins eds. (1984) Transcription and Translation; Immobilized Cells and Enzymes (IRL Press (1986)); Perbal (1984) A Practical Guide to Molecular Cloning; Miller and Calos eds. (1987) Gene Transfer Vectors for Mammalian Cells (Cold Spring Harbor Laboratory); Makrides ed. (2003) Gene Transfer and Expression in Mammalian Cells; Mayer and Walker eds. (1987) Immunochemical Methods in Cell and Molecular Biology (Academic Press, London); Herzenberg et al. eds (1996) Weir's Handbook of Experimental Immunology; Manipulating the Mouse Embryo: A Laboratory Manual, 3^{rd} edition (Cold Spring Harbor Laboratory Press (2002)); Sohail (ed.) (2004) Gene Silencing by RNA Interference: Technology and Application (CRC Press).

All numerical designations, e.g., pH, temperature, time, concentration, and molecular weight, including ranges, are approximations which are varied (+) or (−) by increments of 0.1 or 1.0, where appropriate. It is to be understood, although not always explicitly stated that all numerical designations are preceded by the term “about.” It also is to be understood, although not always explicitly stated, that the reagents described herein are merely exemplary and that equivalents of such are known in the art.

As used in the specification and claims, the singular form “a”, “an” and “the” include plural references unless the context clearly dictates otherwise. For example, the term “a cell” includes a plurality of cells, including mixtures thereof.

Unless specifically stated or obvious from context, as used herein, the term “or” is understood to be inclusive.

The term “including” is used herein to mean, and is used interchangeably with, the phrase “including but not limited to.”

As used herein, the term “comprising” or “comprises” is intended to mean that the compositions and methods include the recited elements, but not excluding others. “Consisting essentially of” when used to define compositions and methods, shall mean excluding other elements of any essential significance to the combination for the stated purpose. Thus, a composition consisting essentially of the elements as defined herein would not exclude trace contaminants from the isolation and purification method and pharmaceutically acceptable carriers, such as phosphate buffered saline, preservatives and the like. “Consisting of” shall mean excluding more than trace elements of other ingredients and substantial method steps for administering the compositions of this invention or process steps to produce a composition or achieve an intended result. Embodiments defined by each of these transition terms are within the scope of this invention.

The terms “glucocerebrosidase 1” and “GBA1” and “GBA1 polypeptide” are used interchangeably to refer to a β-glucocerebrosidase protein or polypeptide that catalyzes the cleavage of beta-glucosidic linkage of glycosphingolipid glucocerebroside (glucosylceramide, GlcCer) to glucose and ceramide. GBA1 is also known as acid β-glucosidase; D-glucosyl-N-acylsphingosine glucohydrolase; GCase; and glucosidase, beta, acid, and transcript variant 1.

The terms “glucocerebrosidase 1 gene” and “GBA1 gene” and “GBA1” are used interchangeably to refer to a nucleic acid or polynucleotide that encodes a β-glucocerebrosidase protein or polypeptide. Mutations in this gene can cause Gaucher disease, a lysosomal storage disease characterized by an accumulation of glucocerebrosides and glucosylsphingosines. Information regarding GBA can be found in the Entrez Gene database at GeneID: 2629.

The term “glucocerebrosidase activity” refers to the cleavage of glucocerebroside.

The terms “polynucleotide,” “nucleic acid” and “oligonucleotide” are used interchangeably and refer to a polymeric form of nucleotides of any length, either deoxyribonucleotides or ribonucleotides or analogs thereof. Polynucleotides can have any three-dimensional structure and may perform any function, known or unknown. The following are non-limiting examples of polynucleotides: a gene or gene fragment (for example, a probe, primer, EST or SAGE tag), exons, introns, messenger RNA (mRNA), transfer RNA, ribosomal RNA, ribozymes, cDNA, recombinant polynucleotides, branched polynucleotides, plasmids, vectors, isolated DNA of any sequence, isolated RNA of any sequence, nucleic acid probes and primers. A polynucleotide can comprise modified nucleotides, such as methylated nucleotides and nucleotide analogs. If present, modifications to the nucleotide structure can be imparted before or after assembly of the polynucleotide. The sequence of nucleotides can be interrupted by non-nucleotide components. A polynucleotide can be further modified after polymerization, such as by conjugation with a labeling component. The term also refers to both double- and single-stranded molecules. Unless otherwise specified or required, any embodiment of this invention that is a polynucleotide encompasses both the double-stranded form and each of two complementary single-stranded forms known or predicted to make up the double-stranded form.

A polynucleotide is composed of a specific sequence of four nucleotide bases: adenine (A); cytosine (C); guanine (G); thymine (T); and uracil (U) for thymine when the polynucleotide is RNA. Thus, the term “polynucleotide sequence” is the alphabetical representation of a polynucleotide molecule. This alphabetical representation can be input into databases in a computer having a central processing unit and used for bioinformatics applications such as functional genomics and homology searching.

The terms “polypeptide” and “protein” are used interchangeably to refer to a polymer of amino acid residues, and are not limited to a minimum length. Such polymers of amino acid residues may contain natural or non-natural amino acid residues, and include, but are not limited to, peptides, oligopeptides, dimers, trimers, and multimers of amino acid residues. Both full-length proteins and fragments thereof are encompassed by the definition. The terms also include post-expression modifications of the polypeptide, for example, glycosylation, sialylation, acetylation, phosphorylation, and the like. Furthermore, for purposes of the present invention, a “polypeptide” refers to a protein which includes modifications, such as deletions, additions, and substitutions (generally conservative in nature), to the native sequence, as long as the protein maintains the desired activity. These modifications may be deliberate, as through site-directed mutagenesis, or may be accidental, such as through mutations of hosts which produce the proteins or errors due to PCR amplification.

“Homology” or “identity” or “similarity” refers to sequence similarity between two peptides or between two nucleic acid molecules. Homology can be determined by comparing a position in each sequence which may be aligned for purposes of comparison. When a position in the compared sequence is occupied by the same base or amino acid, then the molecules are homologous at that position. A degree of homology between sequences is a function of the number of matching or homologous positions shared by the sequences. An “unrelated” or “non-homologous” sequence shares less than 40% identity, or alternatively less than 25% identity, with one of the sequences of the present invention.

A polynucleotide or polynucleotide region (or a polypeptide or polypeptide region) has a certain percentage (for example, 70%, 75%, 80%, 85%, 90%, 95%, 98% or 99%) of “sequence identity” to another sequence means that, when aligned, that percentage of bases (or amino acids) are the same in comparing the two sequences. This alignment and the percent homology or sequence identity can be determined using software programs known in the art, for example those described in Ausubel et al. eds. (2007) Current Protocols in Molecular Biology. Default parameters can be used for alignment. One alignment program is BLAST, using default parameters. Exemplary programs include, but are not limited to, BLASTN and BLASTP, using the following default parameters: Genetic code=standard; filter=none; strand=both; cutoff=60; expect=10; Matrix=BLOSUM62; Descriptions=50 sequences; sort by=HIGH SCORE; Databases=non-redundant, GenBank+EMBL+DDBJ+PDB+GenBank CDS translations+SwissProtein+SPupdate+PIR. Details of these programs can be found at the following Internet address: http://www.ncbi.nlm.nih.gov/cgi-bin/BLAST.

An equivalent nucleic acid, polynucleotide or oligonucleotide is one having at least 80% sequence identity, or alternatively at least 85% sequence identity, or alternatively at least 90% sequence identity, or alternatively at least 92% sequence identity, or alternatively at least 95% sequence identity, or alternatively at least 97% sequence identity, or alternatively at least 98% sequence identity to the reference nucleic acid, polynucleotide, or oligonucleotide.

A “gene” refers to a polynucleotide containing at least one open reading frame (ORF) that is capable of encoding a particular polypeptide or protein after being transcribed and translated.

The term “express” refers to the production of a gene product.

As used herein, “expression” refers to the process by which polynucleotides are transcribed into mRNA and/or the process by which the transcribed mRNA is subsequently being translated into peptides, polypeptides, or proteins. If the polynucleotide is derived from genomic DNA, expression may include splicing of the mRNA in a eukaryotic cell.

A “gene product” or alternatively a “gene expression product” refers to the amino acid (e.g., peptide or polypeptide) generated when a gene is transcribed and translated.

“Heterologous” means derived from a genotypically distinct entity from that of the rest of the entity to which it is compared or into which it is introduced or incorporated. For example, a polynucleotide introduced by genetic engineering techniques into a different cell type is a heterologous polynucleotide (and, when expressed, can encode a heterologous polypeptide). Similarly, a cellular sequence (e.g., a gene or portion thereof) that is incorporated into a viral vector is a heterologous nucleotide sequence with respect to the vector.

The term “transgene” refers to a polynucleotide that is introduced into a cell and is capable of being transcribed into RNA and optionally, translated and/or expressed under appropriate conditions. In aspects, it confers a desired property to a cell into which it was introduced, or otherwise leads to a desired therapeutic or diagnostic outcome.

“Regulates expression of” is a term well understood in the art and indicates that transcription of a polynucleotide sequence, usually a DNA sequence, depends on its being operatively linked to an element which contributes to the initiation of, or promotes, transcription. “Operatively linked” intends the polynucleotides are arranged in a manner that allows them to function in a cell. In one aspect, this invention provides promoters operatively linked to the downstream sequences, e.g., glucocerebrosidase 1 (GBA1).

The term “encode” as it is applied to polynucleotides refers to a polynucleotide which is said to “encode” a polypeptide if, in its native state or when manipulated by methods well known to those skilled in the art, it can be transcribed and/or translated to produce the mRNA for the polypeptide and/or a fragment thereof. The antisense strand is the complement of such a nucleic acid, and the encoding sequence can be deduced therefrom.

“Detectable labels” or “markers” include, but are not limited to radioisotopes, fluorochromes, chemiluminescent compounds, dyes, and proteins, including enzymes. Detectable labels can also be attached to a polynucleotide, polypeptide, antibody or composition described herein.

“Hybridization” refers to a reaction in which one or more polynucleotides react to form a complex that is stabilized via hydrogen bonding between the bases of the nucleotide residues. The hydrogen bonding may occur by Watson-Crick base pairing, Hoogstein binding, or in any other sequence-specific manner. The complex may comprise two strands forming a duplex structure, three or more strands forming a multi-stranded complex, a single self-hybridizing strand, or any combination of these. A hybridization reaction may constitute a step in a more extensive process, such as the initiation of a PCR reaction, or the enzymatic cleavage of a polynucleotide by a ribozyme.

Hybridization reactions can be performed under conditions of different “stringency”. In general, a low stringency hybridization reaction is carried out at about 40° C. in 10×SSC or a solution of equivalent ionic strength/temperature. A moderate stringency hybridization is typically performed at about 50° C. in 6×SSC, and a high stringency hybridization reaction is generally performed at about 60° C. in 1×SSC. Hybridization reactions can also be performed under “physiological conditions” which is well known to one of skill in the art. A non-limiting example of a physiological condition is the temperature, ionic strength, pH and concentration of Mg²⁺ normally found in a cell.

When hybridization occurs in an antiparallel configuration between two single-stranded polynucleotides, the reaction is called “annealing” and those polynucleotides are described as “complementary”. A double-stranded polynucleotide can be “complementary” or “homologous” to another polynucleotide, if hybridization can occur between one of the strands of the first polynucleotide and the second. “Complementarity” or “homology” (the degree that one polynucleotide is complementary with another) is quantifiable in terms of the proportion of bases in opposing strands that are expected to form hydrogen bonding with each other, according to generally accepted base-pairing rules.

As used herein, the term “vector” refers to a non-chromosomal nucleic acid comprising an intact replicon such that the vector may be replicated when placed within a cell, for example by a process of transformation. Vectors may be viral or non-viral. Viral vectors include retroviruses, adenoviruses, herpesvirus, baculoviruses, modified baculoviruses, papovirus, or otherwise modified naturally occurring viruses. Exemplary non-viral vectors for delivering nucleic acid include naked DNA; DNA complexed with cationic lipids, alone or in combination with cationic polymers; anionic and cationic liposomes; DNA-protein complexes and particles comprising DNA condensed with cationic polymers such as heterogeneous polylysine, defined-length oligopeptides, and polyethylene imine, in some cases contained in liposomes; and the use of ternary complexes comprising a virus and polylysine-DNA.

A “viral vector” is defined as a recombinantly produced virus or viral particle that comprises a polynucleotide to be delivered into a host cell, either in vivo, ex vivo or in vitro. Examples of viral vectors include retroviral vectors, lentiviral vectors, adenovirus vectors, adeno-associated virus vectors, herpes simplex virus vectors, alphavirus vectors and the like. Alphavirus vectors, such as Semliki Forest virus-based vectors and Sindbis virus-based vectors, have also been developed for use in gene therapy and immunotherapy. See Schlesinger and Dubensky (1999) Curr. Opin. Biotechnol. 5:434-439 and Ying et al. (1999) Nat. Med. 5(7):823-827.

As is known to those of skill in the art, there are 6 classes of viruses. The DNA viruses constitute classes I and II. The RNA viruses and retroviruses make up the remaining classes. Class III viruses have a double-stranded RNA genome. Class IV viruses have a positive single-stranded RNA genome, the genome itself acting as mRNA Class V viruses have a negative single-stranded RNA genome used as a template for mRNA synthesis. Class VI viruses have a positive single-stranded RNA genome but with a DNA intermediate not only in replication but also in mRNA synthesis. Retroviruses carry their genetic information in the form of RNA; however, once the virus infects a cell, the RNA is reverse-transcribed into the DNA form which integrates into the genomic DNA of the infected cell. The integrated DNA form is called a provirus.

That the vector particle according to the invention is “based on” a particular virus means that the vector is derived from that particular virus. The genome of the vector particle comprises components from that virus as a backbone. The vector particle contains essential vector components compatible with the viral genome. Although some of the structural components of the vector particle will normally be derived from that virus certain components may originate from a different virus (e.g., structural components to give the vector particle a different specificity).

The term “promoter” refers to a region of DNA that initiates transcription of a particular gene. The promoter includes the core promoter, which is the minimal portion of the promoter required to properly initiate transcription and can also include regulatory elements such as transcription factor binding sites. The regulatory elements may promote transcription or inhibit transcription. Regulatory elements in the promoter can be binding sites for transcriptional activators or transcriptional repressors. A promoter can be constitutive or inducible. A constitutive promoter refers to one that is always active and/or constantly directs transcription of a gene above a basal level of transcription. An inducible promoter is one which is capable of being induced by a molecule or a factor added to the cell or expressed in the cell. An inducible promoter may still produce a basal level of transcription in the absence of induction, but induction typically leads to significantly more production of the protein. Promoters can also be tissue specific. A tissue specific promoter allows for the production of a protein in a certain population of cells that have the appropriate transcriptional factors to activate the promoter.

An “inverted terminal repeat” or “ITR” sequence is a term well understood in the art and refers to relatively short sequences found at the termini of viral genomes which are in opposite orientation.

An “adeno-associated virus (AAV) inverted terminal repeat (ITR)” sequence, a term well-understood in the art, is an approximately 145-nucleotide sequence that is present at both termini of the native single-stranded AAV genome. The outermost 125 nucleotides of the ITR can be present in either of two alternative orientations, leading to heterogeneity between different AAV genomes and between the two ends of a single AAV genome. The outermost 125 nucleotides also contains several shorter regions of self-complementarity (designated A, A′, B, B′, C, C′ and D regions), allowing intrastrand base-pairing to occur within this portion of the ITR.

A “terminal resolution sequence” or “trs” is a sequence in the D region of the AAV ITR that is cleaved by AAV rep proteins during viral DNA replication. A mutant terminal resolution sequence is refractory to cleavage by AAV rep proteins.

The term “antibody” means an immunoglobulin molecule that recognizes and specifically binds to a target, such as a protein, polypeptide, peptide, carbohydrate, polynucleotide, lipid, or combinations of the foregoing through at least one antigen recognition site within the variable region of the immunoglobulin molecule. As used herein, the term “antibody” encompasses intact polyclonal antibodies, intact monoclonal antibodies, antibody fragments (such as Fab, Fab′, F(ab′)2, Fd, and Fv fragments), single chain Fv (scFv) mutants, multispecific antibodies such as bispecific antibodies generated from at least two intact antibodies, chimeric antibodies, humanized antibodies, human antibodies, fusion proteins comprising an antigen determination portion of an antibody, and any other modified immunoglobulin molecule comprising an antigen recognition site so long as the antibodies exhibit the desired biological activity. An antibody can be of any the five major classes of immunoglobulins: IgA, IgD, IgE, IgG, and IgM, or subclasses (isotypes) thereof (e.g. IgG1, IgG2, IgG3, IgG4, IgA1 and IgA2), based on the identity of their heavy-chain constant domains referred to as alpha, delta, epsilon, gamma, and mu, respectively. The different classes of immunoglobulins have different and well known subunit structures and three-dimensional configurations. Antibodies can be naked or conjugated to other molecules such as toxins, radioisotopes, and the like.

The term “antibody fragment” refers to a portion of an intact antibody and refers to the antigenic determining variable regions of an intact antibody. Examples of antibody fragments include, but are not limited to Fab, Fab′, F(ab′)2, Fd, and Fv fragments, linear antibodies, single chain antibodies, and multispecific antibodies formed from antibody fragments.

A “monoclonal antibody” refers to homogenous antibody population involved in the highly specific recognition and binding of a single antigenic determinant, or epitope. This is in contrast to polyclonal antibodies that typically include different antibodies directed against different antigenic determinants. The term “monoclonal antibody” encompasses both intact and full-length monoclonal antibodies as well as antibody fragments (such as Fab, Fab′, F(ab′)2, Fd, Fv), single chain (scFv) mutants, fusion proteins comprising an antibody portion, and any other modified immunoglobulin molecule comprising an antigen recognition site. Furthermore, “monoclonal antibody” refers to such antibodies made in any number of manners including but not limited to by hybridoma, phage selection, recombinant expression, and transgenic animals.

The term “humanized antibody” refers to forms of non-human (e.g., murine) antibodies that are specific immunoglobulin chains, chimeric immunoglobulins, or fragments thereof that contain minimal non-human (e.g., murine) sequences. Typically, humanized antibodies are human immunoglobulins in which residues from the complementary determining region (CDR) are replaced by residues from the CDR of a non-human species (e.g., mouse, rat, rabbit, hamster) that have the desired specificity, affinity, and capability (see Jones et al. (1986) Nature 321:522-525; Riechmann et al. (1988) Nature 332:323-327; and Verhoeyen et al. (1988) Science 239: 1534-1536). In some instances, the Fv framework region (FR) residues of a human immunoglobulin are replaced with the corresponding residues in an antibody from a non-human species that has the desired specificity, affinity, and capability. The humanized antibody can be further modified by the substitution of additional residue either in the Fv framework region and/or within the replaced non-human residues to refine and optimize antibody specificity, affinity, and/or capability. In general, the humanized antibody will comprise substantially all of at least one, and typically two or three, variable domains containing all or substantially all of the CDR regions that correspond to the non-human immunoglobulin whereas all or substantially all of the FR regions are those of a human immunoglobulin consensus sequence. The humanized antibody can also comprise at least a portion of an immunoglobulin constant region or domain (Fc), typically that of a human immunoglobulin. Examples of methods used to generate humanized antibodies are described in U.S. Pat. No. 5,225,539.

The term “human antibody” means an antibody produced by a human or an antibody having an amino acid sequence corresponding to an antibody produced by a human made using any technique known in the art. This definition of a human antibody includes intact or full-length antibodies, fragments thereof, and/or antibodies comprising at least one human heavy and/or light chain polypeptide such as, for example, an antibody comprising murine light chain and human heavy chain polypeptides.

The term “chimeric antibodies” refers to antibodies wherein the amino acid sequence of the immunoglobulin molecule is derived from two or more species. Typically, the variable region of both light and heavy chains corresponds to the variable region of antibodies derived from one species of mammals (e.g., mouse, rat, rabbit, etc.) with the desired specificity, affinity, and capability while the constant regions are homologous to the sequences in antibodies derived from another (usually human) to avoid eliciting an immune response in that species.

The term “epitope” or “antigenic determinant” are used interchangeably herein and refer to that portion of an antigen capable of being recognized and specifically bound by a particular antibody. When the antigen is a polypeptide, epitopes can be formed both from contiguous amino acids and noncontiguous amino acids juxtaposed by tertiary folding of a protein. Epitopes formed from contiguous amino acids are typically retained upon protein denaturing, whereas epitopes formed by tertiary folding are typically lost upon protein denaturing. An epitope typically includes at least 3, at least 5, or at least 8-10 amino acids in a unique spatial conformation.

That an antibody “specifically binds” to an epitope or antigenic molecule means that the antibody reacts or associates more frequently, more rapidly, with greater duration, with greater affinity, or with some combination of the above to an epitope or antigenic molecule than with alternative substances, including unrelated proteins. In embodiments, “specifically binds” means, for instance, that an antibody binds to a protein with a KD of about 0.1 mM or less, but more usually less than about 1 μM. In embodiments, “specifically binds” means that an antibody binds to a protein at times with a KD of at least about 0.1 μM or less, and at other times at least about 0.01 μM or less. Because of the sequence identity between homologous proteins in different species, specific binding can include an antibody that recognizes a particular protein in more than one species. It is understood that an antibody or binding moiety that specifically binds to a first target may or may not specifically bind to a second target. As such, “specific binding” does not necessarily require (although it can include) exclusive binding, e.g., binding to a single target. Generally, but not necessarily, reference to binding means specific binding.

The term “proteinopathy” refers to a disease in which certain proteins become structurally abnormal and/or accumulate in a toxic manner, and thereby disrupt the function of cells, tissues and organs of the body. Often the proteins fail to fold into their normal configuration. In this misfolded state, the proteins can become toxic or can lose their normal function. Non-limiting examples of proteinopathies include Alzheimer's disease, Gaucher disease, frontotemporal dementia, progressive supranuclear palsy, dementia pugilistica, Parkinsonism, Parkinson's disease, dementia with Lewy bodies, Pick's disease, corticobasal degeneration, Argyrophilic grain disease, ganglioglioma and gangliocytoma, meningioangiomatosis, subacute sclerosing panencephalitis, lead encephalopathy, tuberous sclerosis, Hallervorden-Spatz disease, and lipofuscinosis, cerebral β-amyloid angiopathy, retinal ganglion cell degeneration in glaucoma, prion diseases, amyotrophic lateral sclerosis (ALS), Huntington's disease and other triplet repeat disorders, Alexander disease, seipinopathies, amyloidotic neuropathy, senile systemic amyloidosis, serpinopathies, amyloidosis, inclusion body myositis/myopathy, Mallory bodies, pulmonary alveolar proteinosis, and critical illness myopathy (CIM).

A “subject,” “individual” or “patient” is used interchangeably herein, and refers to a vertebrate, such as a mammal Mammals include, but are not limited to, murines, rats, rabbit, simians, bovines, ovine, porcine, canines, feline, farm animals, sport animals, pets, equine, primates, and humans. In embodiments, the mammals include horses, dogs, and cats. In another embodiment of the present invention, the mammal is a human patient.

“Administering” is defined herein as a means of providing an agent or a composition containing the agent to a subject in a manner that results in the agent being inside the subject's body. Such an administration can be by any route including, without limitation, oral, transdermal (e.g., vagina, rectum, oral mucosa), by injection (e.g., subcutaneous, intravenous, parenterally, intraperitoneally, into the CNS), or by inhalation (e.g., oral or nasal). Pharmaceutical preparations are, of course, given by forms suitable for each administration route.

“Treating” or “treatment” of a disease includes: (1) preventing the disease, i.e., causing the clinical symptoms of the disease not to develop in a patient that may be predisposed to the disease but does not yet experience or display symptoms of the disease; (2) inhibiting the disease, i.e., arresting or reducing the development of the disease or its clinical symptoms; or (3) relieving the disease, i.e., causing regression of the disease or its clinical symptoms.

The term “suffering” as it related to the term “treatment” refers to a patient or individual who has been diagnosed with or is predisposed to the disease. A patient may also be referred to being “at risk of suffering” from a disease because of a history of disease in their family lineage or because of the presence of genetic mutations associated with the disease. This patient has not yet developed all or some of the characteristic disease pathology.

An “effective amount” or “therapeutically effective amount” is an amount sufficient to effect beneficial or desired results. An effective amount can be administered in one or more administrations, applications or dosages. Such delivery is dependent on a number of variables including the time period for which the individual dosage unit is to be used, the bioavailability of the therapeutic agent, the route of administration, etc. It is understood, however, that specific dose levels of the therapeutic agents of the present invention for any particular subject depends upon a variety of factors including the activity of the specific compound employed, the age, body weight, general health, sex, and diet of the subject, the time of administration, the rate of excretion, the drug combination, and the severity of the particular disorder being treated and form of administration. Treatment dosages generally may be titrated to optimize safety and efficacy. Typically, dosage-effect relationships from in vitro and/or in vivo tests initially can provide useful guidance on the proper doses for patient administration. In general, one will desire to administer an amount of the compound that is effective to achieve a serum level commensurate with the concentrations found to be effective in vitro. Determination of these parameters is well within the skill of the art. These considerations, as well as effective formulations and administration procedures are well known in the art and are described in standard textbooks. Consistent with this definition, as used herein, the term “therapeutically effective amount” is an amount sufficient to augment glucocerebrosidase activity to treat (e.g., improve) one or more symptoms associated with proteinopathy or aberrant/increased levels of toxic lipids, α-synuclein, tau, or protein aggregates ex vivo, in vitro or in vivo.

As used herein, the term “pharmaceutically acceptable carrier” encompasses any of the standard pharmaceutical carriers, such as a phosphate buffered saline solution, water, and emulsions, such as an oil/water or water/oil emulsion, and various types of wetting agents. The compositions also can include stabilizers and preservatives. For examples of carriers, stabilizers and adjuvants, see Remington's Pharmaceutical Sciences (20th ed., Mack Publishing Co. 2000).

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50.

The recitation of a listing of chemical groups in any definition of a variable herein includes definitions of that variable as any single group or combination of listed groups. The recitation of an embodiment for a variable or aspect herein includes that embodiment as any single embodiment or in combination with any other embodiments or portions thereof.

Any compositions or methods provided herein can be combined with one or more of any of the other compositions and methods provided herein.

### DESCRIPTIVE EMBODIMENTS

This disclosure relates to methods and compositions for treating proteinopathies. Increasing glucocerebrosidase in a mammal has therapeutically beneficial outcomes such as improving neural function, improving memory function, preventing loss of memory or neural function, reducing toxic lipids (e.g., glucosylsphingosine), reducing α-synuclein, reducing tau, and inhibiting the accumulation of protein aggregates. In one embodiment, the improvement of neural function is observed in subjects exhibiting a reduction in memory function due to a proteinopathy. Diagnosis of a cognitive impairment is within the routine skill of a medical practitioner. Cognitive tests are known in the art and can include tests such as the abbreviated mental test score (AMTS), the mini mental state examination (MMSE), informant questionnaire on cognitive decline in the elderly (IQCODE), and the General Practitioner Assessment of Cognition that test for cognitive impairment. These tests can assess impairments in, for example, memory, reasoning skills, problem solving skills, decision making skills, attention span, and language skills. Imaging methods are also available to diagnose cognitive decline. For example, the functional neuroimaging modalities of single-photon emission computed tomography (SPECT) and positron emission tomography (PET), are useful in assessing cognitive dysfunction. In some aspects, the improvement of neural function is measured by evaluating the memory function or cognitive function of the patient.

Relating to methods for preventing cognitive decline, such as memory loss, PET imaging using carbon-11 Pittsburgh Compound B as a radiotracer (PIB-PET) has been useful in predictive diagnosis of various kinds of proteinopathies. For example, studies have found PIB-PET to be 86% accurate in predicting which patients with mild cognitive impairment would develop Alzheimer's disease within two years. In another study, using either PIB or another radiotracer, carbon-11 dihydrotetrabenazine (DTBZ), led to more accurate diagnosis for more than one-fourth of patients with mild cognitive impairment or mild dementia.

In certain embodiments of the methods described herein, the mammal has reduced glucocerebrosidase activity prior to treatment. Glucocerebrosidase activity can be assessed by methods known in the art. For example, the glucocerebrosidase activity may be measured from the cerebral spinal fluid of mammals.

In some embodiments, the mammal is “wild-type” for the GBA1 gene. The term “wild-type” refers to a gene or protein with no detectable sequence mutations known to affect the enzymatic activity of the protein. Such sequences are well known in the art, and nonlimiting examples can be found at GenBank accession numbers NM_{—}000157.3 (mRNA) and NP_{—}000148.2 (protein). An exemplary sequence for a mature GBA1 protein is:

(SEQ ID NO: 1)
ARPCIPKSFGYSSVVCVCNATYCDSFDPPTFPALGTFSRYESTRSGRRME
LSMGPIQANHTGTGLLLTLQPEQKFQKVKGFGGAMTDAAALNILALSPPA
QNLLLKSYFSEEGIGYNIIRVPMASCDFSIRTYTYADTPDDFQLHNFSLP
EEDTKLKIPLIHRALQLAQRPVSLLASPWTSPTWLKTNGAVNGKGSLKGQ
PGDIYHQTWARYFVKFLDAYAEHKLQFWAVTAENEPSAGLLSGYPFQCLG
FTPEHQRDFIARDLGPTLANSTHHNVRLLMLDDQRLLLPHWAKVVLTDPE
AAKYVHGIAVHWYLDFLAPAKATLGETHRLFPNTMLFASEACVGSKFWEQ
SVRLGSWDRGMQYSHSIITNLLYHVVGWTDWNLALNPEGGPNWVRNFVDS
PIIVDITKDTFYKQPMFYHLGHFSKFIPEGSQRVGLVASQKNDLDAVALM
HPDGSAVVVVLNRSSKDVPLTIKDPAVGFLETISPGYSIHTYLWRRQ

When the gene is found to be wild-type, but a reduction in glucocerebrosidase activity is observed, the reduction in activity may be due to suppression of activity of the protein or repression of transcription or translation of the gene/protein. These mechanisms are well known in the art. For example, the production of the protein may be repressed by aberrant cellular mechanism. Alternatively, the protein may be modified in the cell which causes reduced or loss of enzymatic activity.

In some embodiments, the mammal has one or more mutations in the glucocerebrosidase 1 (GBA1) gene. Specific mutations in GBA1 that may affect the activity of the protein include L444P, D409H, D409V, E235A, and E340A (see, for example, Cullen et al. (2011) Annals of Neurology 69:940-953, which is incorporated by reference for all purposes). In a specific embodiment, the mutation is a D409V mutation.

The methods disclosed herein are useful for treating mammals with a proteinopathy. In certain embodiments, the proteinopathy comprises protein aggregates. “Protein aggregation” refers to the biological phenomenon in which misfolded proteins aggregate either intra- or extracellularly. These protein aggregates may be toxic. In certain embodiments, the protein aggregates comprise a protein selected from the group consisting of ubiquitin, tau, and α-synuclein. Ubiquitin is a small protein that is found in almost all tissues of eukaryotic organisms. It is a 76 amino acid protein that can be attached to a substrate protein. Addition of ubiquitin can result in protein degradation; modulation of transcription, translation, and protein localization; or modulation of protein activity/interactions.

The term “tau” refers to tau proteins that function to stabilize microtubules. They are abundant in neurons of the central nervous system and in astrocytes and oligodendrocytes. Hyperphosphorylation of the tau protein (tau inclusions, pTau) can result in the self-assembly of tangles of paired helical filaments and straight filaments, which are involved in the pathogenesis of Alzheimer's disease and other tauopathies.

Certain aspects of the invention relate to methods for treating or preventing tauopathy in a subject (e.g., improving neural function in a mammal with a tauopathy) comprising administering a therapeutically effective amount of an agent that increases glucocerebrosidase activity in the mammal Tauopathies are neurodegenerative disorders characterized by accumulation of tau. Exemplary tauopathies include, but are not limited to, Alzheimer's disease, progressive supranuclear palsy, dementia pugilistica, Parkinson's disease, parkinsonism linked to chromosome 17, Lytico-Bodig disease, tangle-predominant dementia, Argyrophilic grain disease, ganglioglioma, gangliocytoma, meningioangiomatosis, subacute sclerosing panencephalitis, lead encephalopathy, tuberous sclerosis, Hallervorden-Spatz disease, lipofuscinosis, dementia with Lewy bodies, Pick's disease, corticobasal degeneration, frontotemporal dementia, and frontotemporal lobar degeneration. All of the six tau isoforms are present in an often hyperphosphorylated state in paired helical filaments from Alzheimer's disease brain. In other neurodegenerative diseases, the deposition of aggregates enriched in certain tau isoforms has been reported. When misfolded, this otherwise very soluble protein can form extremely insoluble aggregates that contribute to a number of neurodegenerative diseases.

“α-synuclein” is a protein that, in humans, is encoded by the SNCA gene. The protein is found in neural tissue and predominantly expressed in the neocortex, hippocampus, substantia nigra, thalamus, and cerebellum. Besides neurons, the protein can also be found in neuroglial cells and melanocytic cells. α-synuclein can aggregate to form insoluble fibrils in pathological conditions that are, in some instances, characterized by Lewy bodies. In a specific embodiment, the proteinopathy is a synucleinopathy. Non-limiting examples of synucleinopathies include Parkinson's, multiple system atrophy, and Lewy Body dementia. Some diseases classified as synucleinopathies may also have accumulation on the tau protein, and some diseases classified as tauopathies may have also have accumulation of the α-synuclein protein.

In certain embodiments, the proteinopathy recited in the methods disclosed herein is a disease selected from the group consisting of Alzheimer's disease, Gaucher disease, frontotemporal dementia, progressive supranuclear palsy, Parkinsonism, Parkinson's disease, Lytico-Bodig disease, dementia with Lewy bodies, tangle-predominant dementia, dementia pugilistica, Pick's disease, corticobasal degeneration, Argyrophilic grain disease, ganglioglioma and gangliocytoma, meningioangiomatosis, subacute sclerosing panencephalitis, lead encephalopathy, tuberous sclerosis, Hallervorden-Spatz disease, and lipofuscinosis.

The agent used in the methods described herein can be an agent that increases glucocerebrosidase activity in mammals. For example, the agent can be any small molecule compound, antibody, nucleic acid molecule, polypeptide, or biological equivalent thereof that increases glucocerebrosidase activity in mammals.

In aspects, the agent comprises a nucleic acid encoding a GBA1 gene or biological equivalent thereof (e.g., fragment, analog, or derivative thereof that encodes a polypeptide that catalyzes the cleavage of glucocerebroside). A biological equivalent of the nucleic acid can be a naturally occurring allelic variant of the polynucleotide or a non-naturally occurring variant of the polynucleotide. In certain embodiments, the nucleic acid can have a coding sequence which is an allelic variant of the coding sequence of a GBA1 polypeptide disclosed herein. As known in the art, an allelic variant is an alternate form of a polynucleotide sequence that have, for example, a substitution, deletion, or addition of one or more nucleotides, which does not substantially alter the function of the encoded polypeptide.

In embodiments, the biological equivalent of GBA1 is one that comprises the minimal sequences required for glucocerebrosidase enzyme activity. In another embodiment, the biological equivalent thereof comprises a nucleic acid that hybridizes under conditions of high stringency to the complement of a GBA1 polynucleotide described herein (e.g., a polynucleotide that encodes the GBA1 amino acid sequence disclosed herein). In another embodiment, the biological equivalent thereof comprises a nucleic acid having at least 80% sequence identity, or alternatively at least 85% sequence identity, or alternatively at least 90% sequence identity, or alternatively at least 92% sequence identity, or alternatively at least 95% sequence identity, or alternatively at least 97% sequence identity, or alternatively at least 98% sequence identity to a GBA1 polynucleotide described herein (e.g., a polynucleotide that encodes the GBA1 amino acid sequence disclosed herein).

In embodiments, the nucleic acid contains a coding sequence for the mature GBA1 polypeptide or a biological equivalent thereof fused in the same reading frame to a polynucleotide which aids, for example, in expression and secretion of a polypeptide from a host cell (e.g., a leader sequence which functions as a secretory sequence for controlling transport of a polypeptide from the cell). The polypeptide having a leader sequence is a preprotein and that is cleaved by the host cell to generate the mature form of the polypeptide. The polynucleotides can also encode for a proprotein which is the mature protein plus additional 5′ amino acid residues. A mature protein having a prosequence is a proprotein and is an inactive form of the protein. Once the prosequence is cleaved an active mature protein remains.

In embodiments, the nucleic acid contains a marker sequence that allows, for example, detection or purification of the encoded polypeptide. Such markers are well known in the art and an overview of exemplary markers can be found in Michael R. Green and Joseph Sambrook, Molecular Cloning (4^{th} ed., Cold Spring Harbor Laboratory Press 2012). Exemplary markers include, but are not limited, to histidine tags; hemagglutinin (HA) tags; Calmodulin tags; FLAG tags; Myc tags; S tags; SBP tags; Softag 1; Softag 3; V5 tags; Xpress tags; Isopeptag; SpyTag; Biotin Carboxyl Carrier Protein (BCCP) tags; GST tags; fluorescent protein tags such as enhanced green fluorescent protein (EGFP), red fluorescence protein (RFP), green fluorescent protein (GFP), yellow fluorescent protein (YFP), and the like, maltose binding protein tags, Nus tags, Strep-tags, thioredoxin tags, TC tags, and Ty tags.

The nucleic acids described herein can be produced by any suitable method known in the art. In embodiments, the nucleic acid is constructed by chemical synthesis using an oligonucleotide synthesizer. In embodiments, DNA oligomers containing a nucleotide sequence coding for a particular polypeptide can be synthesized and then ligated. The individual oligonucleotides typically contain 5′ or 3′ overhangs for complementary assembly.

Once assembled, the polynucleotide sequences can be inserted into an expression vector and optionally operatively linked to an expression control sequence appropriate for expression of the protein in a desired host. The polynucleotide can also be delivered to a cell (e.g., in vivo or in vitro) using non-vector based delivery methods. See, e.g., Yuan, Non-Viral Gene Therapy (InTech 2011). Proper assembly can be confirmed by nucleotide sequencing, restriction mapping, expression of a biologically active polypeptide in a suitable host, and the like.

Nucleic acids may be delivered to the cell by a variety of mechanisms commonly known to those of skill in the art. Viral constructs can be delivered through the production of a virus in a suitable host cell. Virus is then harvested from the host cell and contacted with the target cell. Viral and non-viral vectors capable of expressing genes of interest can be delivered to a targeted cell via DNA/liposome complexes, micelles and targeted viral protein-DNA complexes. Liposomes that also comprise a targeting antibody or fragment thereof can be used in the methods of this invention. In addition to the delivery of polynucleotides to a cell or cell population, direct introduction of the proteins described herein to the cell or cell population can be done by the non-limiting technique of protein transfection, alternatively culturing conditions that can enhance the expression and/or promote the activity of the proteins of this invention are other non-limiting techniques.

Other methods of delivering vectors encoding genes of the current invention include but are not limited to, calcium phosphate transfection, DEAE-dextran transfection, electroporation, microinjection, protoplast fusion, or liposome-mediated transfection. The host cells that are transfected with the vectors of this invention may include (but are not limited to) E. coli or other bacteria, yeast, fungi, insect cells (using, for example, baculoviral vectors for expression in SF9 insect cells), or cells derived from mice, humans, or other animals (e.g., mammals). In vitro expression of a protein, fusion, polypeptide fragment, or mutant encoded by cloned DNA may also be used. Those skilled in the art of molecular biology will understand that a wide variety of expression systems and purification systems may be used to produce recombinant proteins and fragments thereof.

In aspects, the agent is a non-viral vector comprising a heterologous polynucleotide capable of being delivered to a target cell, either in vitro, in vivo or ex-vivo. The heterologous polynucleotide can comprise a sequence of interest and can be operably linked to one or more regulatory elements and may control the transcription of the nucleic acid sequence of interest. As used herein, a vector need not be capable of replication in the ultimate target cell or subject. The term vector may include expression vector and cloning vector.

The promoter that regulates expression of the nucleic acid encoding the GBA1 gene or equivalent thereof can be a constitutive, inducible, or tissue specific promoter. In certain embodiments, inducible systems may be used when constructing promoters. Non-limiting examples of inducible systems include regulation by tetracycline, ecdysone, by estrogen, progesterone, chemical inducers of dimerization, and isopropyl-beta-D1-thiogalactopyranoside (EPTG).

Promoters useful in this disclosure can be constitutive or inducible. Some examples of promoters include SV40 early promoter, mouse mammary tumor virus LTR promoter, adenovirus major late promoter, herpes simplex virus promoter, and the CMV promoter.

In aspects, the agent is a viral vector comprising a nucleic acid encoding a gene of interest (e.g., GBA1 or a biological equivalent thereof). Viral gene transfer is an effective method for the therapeutic gene transfer of genes in mammals. Viral vectors suitable for use in the present invention are well known in the art. In embodiments, the viral vectors are derived from or based on a neurotropic virus (or a combination of neurotropic viruses). Examples of neurotropic viruses include, but are not limited to, adenovirus, adeno-associated virus (AAV), herpes simplex virus, retrovirus, and lentivirus. Methods for making and using such viral vectors are well known in the art and are described in Carol Shoskes Reiss, Neurotropic Viral Infections (Cambridge University Press, 2008); Michael G. Kaplitt and Matthew J. During, Gene Therapy of the Central Nervous System: From Bench to Bedside (Gulf Professional Publishing 2006); Jean-Michel H. Vos, Viruses in Human Gene Therapy (Springer 1995); Andres M. Lozano et al., Textbook of Stereotactic and Functional Neurosurgery (Springer 2009); and Michael R. Green and Joseph Sambrook, Molecular Cloning (4^{th} ed., Cold Spring Harbor Laboratory Press 2012), each of which is incorporated by reference in its entirety.

In embodiments, the viral vector is derived from or based on a wild-type virus. Examples of such, include without limitation, human immunodeficiency virus (HIV), equine infectious anaemia virus (EIAV), simian immunodeficiency virus (SIV) and feline immunodeficiency virus (FIV). Alternatively, it is contemplated that other retrovirus can be used as a basis for a vector backbone such murine leukemia virus (MLV). It will be evident that a viral vector according to the invention need not be confined to the components of a particular virus. The viral vector may comprise components derived from two or more different viruses, and may also comprise synthetic components. Vector components can be manipulated to obtain desired characteristics, such as target cell specificity.

U.S. Pat. No. 6,924,123 discloses that certain retroviral sequence facilitate integration into the target cell genome. This patent teaches that each retroviral genome comprises genes called gag, pol and env which code for virion proteins and enzymes. These genes are flanked at both ends by regions called long terminal repeats (LTRs). The LTRs are responsible for proviral integration, and transcription. They also serve as enhancer-promoter sequences. In other words, the LTRs can control the expression of the viral genes. Encapsidation of the retroviral RNAs occurs by virtue of a psi sequence located at the 5′ end of the viral genome. The LTRs themselves are identical sequences that can be divided into three elements, which are called U3, R and U5. U3 is derived from the sequence unique to the 3′ end of the RNA. R is derived from a sequence repeated at both ends of the RNA, and U5 is derived from the sequence unique to the 5′ end of the RNA. The sizes of the three elements can vary considerably among different retroviruses. For the viral genome. and the site of poly (A) addition (termination) is at the boundary between R and U5 in the right hand side LTR. U3 contains most of the transcriptional control elements of the provirus, which include the promoter and multiple enhancer sequences responsive to cellular and in some cases, viral transcriptional activator proteins.

With regard to the structural genes gag, pol and env themselves, gag encodes the internal structural protein of the virus. Gag protein is proteolytically processed into the mature proteins MA (matrix), CA (capsid) and NC (nucleocapsid). The pol gene encodes the reverse transcriptase (RT), which contains DNA polymerase, associated RNase H and integrase (IN), which mediate replication of the genome.

For the production of viral vector particles, the vector RNA genome can be expressed from a DNA construct encoding it, in a host cell. The components of the particles not encoded by the vector genome can be provided in trans by additional nucleic acid sequences (the “packaging system”, which usually includes either or both of the gag/pol and env genes) expressed in the host cell. The set of sequences required for the production of the viral vector particles may be introduced into the host cell by transient transfection, or they may be integrated into the host cell genome, or they may be provided in a mixture of ways. The techniques involved are known to those skilled in the art.

In embodiments, the viral vector is derived from or based on an adenovirus. Adenoviruses are a relatively well characterized, homogenous group of viruses, including over 50 serotypes. See, e.g., International PCT Application No. WO 95/27071. Adenoviruses are easy to grow and do not require integration into the host cell genome. Recombinant adenovirus derived vectors, e.g., those that reduce the potential for recombination and generation of wild-type virus, have also been constructed. See, e.g., International PCT Application Nos. WO 95/00655 and WO 95/11984.

In embodiments, the viral vector is derived from or based on adeno-associated virus (AAV). In recombinant AAV (rAAV) systems, nucleic acid sequences encoding for a protein of interest (e.g., a GBA1 protein) are packaged into an AAV viral particle. The recombinant viral genome may include any element to establish the expression of the protein, for example, a promoter, a transgene (e.g., a GBA1 transgene), an ITR, a ribosome binding element, terminator, enhancer, selection marker, intron, polyA signal, and/or origin of replication.

In aspects, recombinant AAV particles of the invention can contain a nucleic acid comprising a sequence encoding a GBA1 flanked by one or two ITRs. The nucleic acid is encapsidated in the AAV particle. The AAV particle also comprises capsid proteins. In some embodiments, the nucleic acid comprises the protein coding sequence(s) of interest (e.g., a transgene encoding a GBA1 protein) operatively linked components in the direction of transcription, control sequences including transcription initiation and termination sequences, thereby forming an expression cassette. The expression cassette is flanked on the 5′ and 3′ end by at least one functional AAV ITR sequences. By “functional AAV ITR sequences” it is meant that the ITR sequences function as intended for the rescue, replication and packaging of the AAV virion. See Davidson et al. (2000) PNAS 97:3428-32; Passini et al. (2003) J. Virol. 77:7034-40; and Pechan et al. (2009) Gene Ther. 16:10-16, all of which are incorporated herein in their entirety by reference. For practicing some aspects of the invention, the recombinant vectors comprise at least all of the sequences of AAV essential for encapsidation and the physical structures for infection by the rAAV. AAV ITRs for use in the vectors of the invention need not have a wild-type nucleotide sequence (e.g., as described in Kotin Hum. Gene Ther. (1994) 5:793-801), and may be altered by the insertion, deletion or substitution of nucleotides or the AAV ITRs may be derived from any of several AAV serotypes.

More than 40 serotypes of AAV are currently known, and new serotypes and variants of existing serotypes continue to be identified. See Gao et al. (2002) PNAS 99: 11854-6; Gao et al. (2003) PNAS 100:6081-6; and Bossis et al. (2003) J. Virol. 77:6799-810. Use of any AAV serotype is considered within the scope of the present invention. rAAV vector can be a vector derived from any AAV serotype, including without limitation, AAV1, AAV2, AAV3, AAV4, AAV5, AA6, AAV7, AAV8, AAV9, AAVrh.8, AAVrh.10, AAV11, or AAV12 or the like. The nucleic acid in the AAV can contain an ITR of AAV1, AAV2, AAV3, AAV4, AAV5, AA6, AAV7, AAV8, AAV9, AAVrh.8, AAVrh.10, AAV11, AAV12 or the like, and the rAAV particle can contain capsid proteins of AAV1, AAV2, AAV3, AAV4, AAV5, AA6, AAV7, AAV8, AAV9, AAVrh.8, AAVrh.10, AAV11, AAV12 or the like. The rAAV particle can also contain ITRs or capsid proteins from any AAV serotype from Clades A-F (Gao et al. (2004) J. Virol. 78(12):6381).

Different AAV serotypes can be used to optimize transduction of particular target cells or to target specific cell types within a particular target tissue (e.g., a diseased tissue). A rAAV particle can comprise viral proteins and viral nucleic acids of the same serotype or a mixed serotype (i.e., a pseudotype AAV). Pseudotyped AAV vectors are those that contain the inverted terminal repeats (ITRs) of one AAV serotype and the capsid of a second AAV serotype. For example, a rAAV particle can comprise AAV1 capsid proteins and at least one AAV2 ITR or it can comprise AAV2 capsid proteins and at least one AAV1 ITR. In yet another example, a rAAV particle can comprise capsid proteins from both AAV1 and at least one additional AAV serotype, and further comprise at least one AAV2 ITR. Any combination of AAV serotypes for production of a rAAV particle is provided herein as if each combination had been expressly stated herein.

The AAV particles of the invention can also be viral particles comprising a recombinant self-complementing genome. AAV viral particles with self-complementing genomes and methods of use of self-complementing AAV genomes are described in U.S. Pat. Nos. 6,596,535; 7,125,717; 7,765,583; 7,785,888; 7,790,154; 7,846,729; 8,093,054; and 8,361,457; and Wang Z. et al. (2003) Gene Ther 10:2105-2111, each of which is incorporated herein by reference in its entirety. An rAAV comprising a self-complementing genome, will quickly form a double stranded DNA molecule by virtue of its partially complementing sequences (e.g., complementing coding and/or non-coding strands of a transgene). In embodiments, the invention provides an AAV viral particle comprising an AAV genome, wherein the rAAV genome comprises a first heterologous polynucleotide sequence (e.g., a GBA1 coding strand) and a second heterologous polynucleotide sequence (e.g., a GBA1 noncoding or antisense strand) wherein the first heterologous polynucleotide sequence can form intrastrand base pairs with the second polynucleotide sequence along some or most/all of its length. In embodiments, the first heterologous polynucleotide sequence and a second heterologous polynucleotide sequence are linked by a sequence that facilitates intrastrand base pairing (e.g., a hairpin DNA structure). In embodiments, the first heterologous polynucleotide sequence and a second heterologous polynucleotide sequence are linked by a mutated ITR (e.g., the right ITR). In some related embodiments, the mutated ITR comprises a deletion of the D region comprising the terminal resolution sequence. As a result, on replicating an AAV viral genome, the rep proteins will not cleave the viral genome at the mutated ITR and as such, a recombinant viral genome comprising the following in 5′ to 3′ order will be packaged in a viral capsid: an AAV ITR, the first heterologous polynucleotide sequence including regulatory sequences, the mutated AAV ITR, the second heterologous polynucleotide in reverse orientation to the first heterologous polynucleotide and a third AAV ITR.

Methods for using AAV vectors to produce rAAV particles are well known in the art. See, e.g., U.S. Pat. Nos. 6,566,118; 6,989,264; and 6,995,006. In practicing the invention, host cells for producing rAAV particles include mammalian cells, insect cells, plant cells, microorganisms and yeast. Host cells can also be packaging cells in which the AAV rep and cap genes are stably maintained in the host cell or producer cells in which the AAV vector genome is stably maintained. Exemplary packaging and producer cells are derived from 293, A549 or HeLa cells. AAV vectors are purified and formulated using standard techniques known in the art.

In aspects where the rAAV particles are purified, the term “purified” as used herein includes a preparation of rAAV particles devoid of at least some of the other components that may also be present where the rAAV particles naturally occur or are initially prepared from. Thus, for example, isolated rAAV particles may be prepared using a purification technique to enrich it from a source mixture, such as a culture lysate or production culture supernatant. Enrichment can be measured in a variety of ways, such as, for example, by the proportion of DNase-resistant particles (DRPs) or genome copies (gc) present in a solution, or by infectivity, or it can be measured in relation to a second, potentially interfering substance present in the source mixture, such as contaminants, including production culture contaminants or in-process contaminants, including helper virus, media components, and the like. In embodiments, the virus infects neuronal cells in the mammal. The term “neuronal cell” or “neuron” refers to electrically excitable cells that make up the central and peripheral nervous system. The neurons may be cells within the body of an animal or cells cultured outside the body of an animal. The term “neuronal cell” or “neuron” also refers to established or primary tissue culture cell lines that are derived from neural cells from a mammal or tissue culture cell lines that are made to differentiate into neurons. “Neuron” or “neuronal cell” also refers to any of the above types of cells that have also been modified to express a particular protein either extrachromosomally or intrachromosomally and also refers to transformed neurons such as neuroblastoma cells and support cells within the brain such as glia. Infection of neuronal cells can be accomplished by a variety of mechanisms known in the art. In one embodiment, the virus is administered locally to the CNS. In related embodiments, the virus is administered by intrahippocampal injection, or alternatively, by intrathecal injection.

In aspects, the agent is comprises a GBA1 protein or biological equivalent thereof (e.g., fragment, analog, or derivative thereof that catalyzes the cleavage of glucocerebroside). The GBA1 protein is known and characterized in the art, and exemplary sequences have been provided herein. In embodiments, the agent comprises a polypeptide having glucocerebrosidase activity and having at least 80% sequence identity, at least 85% sequence identity, at least 90% sequence identity, at least 91% sequence identity, at least 92% sequence identity, at least 93% sequence identity, at least 94% sequence identity, at least 95% sequence identity, at least 96% sequence identity, at least 97% sequence identity, at least 98% sequence identity, or at least 99% sequence identity to a GBA1 polypeptide disclosed herein. A biological equivalent can be a polypeptide that maintains the desired glucocerebrosidase activity (e.g., wild type glucocerebrosidase activity).

The polypeptides described herein can be produced by any suitable method known in the art. In embodiments, direct protein synthetic methods are used. In other embodiments, recombinant expression vectors can be used to amplify and express DNA encoding a protein of interest (e.g., a GBA1 protein or a biological equivalent thereof). See Michael R. Green and Joseph Sambrook, Molecular Cloning (4th ed., Cold Spring Harbor Laboratory Press 2012). Recombinant expression vectors are replicable DNA constructs which have synthetic or cDNA-derived DNA fragments encoding the protein of interest operatively linked to suitable transcriptional or translational regulatory elements derived from mammalian, microbial, viral or insect genes. A transcriptional unit generally comprises an assembly of (1) a genetic element or elements having a regulatory role in gene expression, for example, transcriptional promoters or enhancers, (2) a structural or coding sequence which is transcribed into mRNA and translated into protein, and (3) appropriate transcription and translation initiation and termination sequences, as described in detail below. Such regulatory elements can include an operator sequence to control transcription. The ability to replicate in a host, usually conferred by an origin of replication, and a selection gene to facilitate recognition of transformants can additionally be incorporated. DNA regions are operatively linked when they are functionally related to each other. For example, DNA for a signal peptide (secretory leader) is operatively linked to DNA for a polypeptide if it is expressed as a precursor which participates in the secretion of the polypeptide; a promoter is operatively linked to a coding sequence if it controls the transcription of the sequence; or a ribosome binding site is operatively linked to a coding sequence if it is positioned so as to permit translation. Generally, operatively linked means contiguous, and in the case of secretory leaders, means contiguous and in reading frame. Structural elements intended for use in yeast expression systems include a leader sequence enabling extracellular secretion of translated protein by a host cell. Alternatively, where recombinant protein is expressed without a leader or transport sequence, it can include an N-terminal methionine residue. This residue can optionally be subsequently cleaved from the expressed recombinant protein to provide a final product.

The choice of expression control sequence and expression vector will depend upon the choice of host. A wide variety of expression host/vector combinations can be employed. Useful expression vectors for eukaryotic hosts, include, for example, vectors comprising expression control sequences from SV40, bovine papilloma virus, adenovirus and cytomegalovirus. Useful expression vectors for bacterial hosts include known bacterial plasmids, such as plasmids from Escherichia coli, including pCR 1, pBR322, pMB9 and their derivatives, wider host range plasmids, such as M13 and filamentous single-stranded DNA phages.

Suitable host cells for expression of a polypeptide include prokaryotes, yeast, insect or higher eukaryotic cells under the control of appropriate promoters. Prokaryotes include gram negative or gram positive organisms, for example E. coli or bacilli. Higher eukaryotic cells include established cell lines of mammalian origin. Cell-free translation systems could also be employed. Appropriate cloning and expression vectors for use with bacterial, fungal, yeast, and mammalian cellular hosts are well known in the art. See Pouwels et al., Cloning Vectors: A Laboratory Manual (Elsevier Science 1985).

Various mammalian or insect cell culture systems are also advantageously employed to express recombinant protein. Expression of recombinant proteins in mammalian cells can be performed because such proteins are generally correctly folded, appropriately modified and completely functional. Examples of suitable mammalian host cell lines include the COS-7 lines of monkey kidney cells, described by Gluzman (1981) Cell 23:175, and other cell lines capable of expressing an appropriate vector including, for example, L cells, C127, 3T3, Chinese hamster ovary (CHO), HeLa and BHK cell lines. Mammalian expression vectors can comprise nontranscribed elements such as an origin of replication, a suitable promoter and enhancer linked to the gene to be expressed, and other 5′ or 3′ flanking nontranscribed sequences, and 5′ or 3′ nontranslated sequences, such as necessary ribosome binding sites, a polyadenylation site, splice donor and acceptor sites, and transcriptional termination sequences. Baculovirus systems for production of heterologous proteins in insect cells are reviewed by Luckow and Summers (1988) Bio/Technology 6:47.

The proteins produced by a transformed host can be purified according to any suitable method. Such standard methods include chromatography (e.g., ion exchange, affinity and sizing column chromatography, and the like), centrifugation, differential solubility, or by any other standard technique for protein purification. Affinity tags such as hexahistidine, maltose binding domain, influenza coat sequence, glutathione-S-transferase, and the like can be attached to the protein to allow easy purification by passage over an appropriate affinity column. Isolated proteins can also be physically characterized using such techniques as proteolysis, nuclear magnetic resonance and x-ray crystallography. For example, supernatants from systems which secrete recombinant protein into culture media can be first concentrated using a commercially available protein concentration filter, for example, an Amicon or Millipore Pellicon ultrafiltration unit. Following the concentration step, the concentrate can be applied to a suitable purification matrix. Alternatively, an anion exchange resin can be employed, for example, a matrix or substrate having pendant diethylaminoethyl (DEAE) groups. The matrices can be acrylamide, agarose, dextran, cellulose or other types commonly employed in protein purification. Alternatively, a cation exchange step can be employed. Suitable cation exchangers include various insoluble matrices comprising sulfopropyl or carboxymethyl groups. Finally, one or more reverse-phase high performance liquid chromatography (RP-HPLC) steps employing hydrophobic RP-HPLC media, e.g., silica gel having pendant methyl or other aliphatic groups, can be employed. Some or all of the foregoing purification steps, in various combinations, can also be employed to provide a homogeneous recombinant protein. Recombinant protein produced in bacterial culture can be isolated, for example, by initial extraction from cell pellets, followed by one or more concentration, salting-out, aqueous ion exchange or size exclusion chromatography steps. High performance liquid chromatography (HPLC) can be employed for final purification steps. Microbial cells employed in expression of a recombinant protein can be disrupted by any convenient method, including freeze-thaw cycling, sonication, mechanical disruption, or use of cell lysing agents.

In aspects, the agent comprises an antibody or fragment thereof that specifically binds and enhances the activity of GBA1.

The term “antibody” encompasses full-sized antibodies as well as antigen-binding fragments, variants, analogs, or derivatives of such antibodies, e.g., naturally occurring antibody or immunoglobulin molecules or engineered antibody molecules or fragments that bind antigen in a manner similar to antibody molecules.

An antibody comprises at least the variable domain of a heavy chain, and normally comprises at least the variable domains of a heavy chain and a light chain. Basic immunoglobulin structures in vertebrate systems are well understood. See, e.g., Harlow et al., Antibodies: A Laboratory Manual, (2nd ed., Cold Spring Harbor Laboratory Press 1988), which is hereby incorporated by reference in its entirety.

Antibodies or antigen-binding fragments, variants, or derivatives thereof of the invention include, but are not limited to, human, humanized, primatized, or chimeric antibodies, single chain antibodies, epitope-binding fragments, e.g., Fab, Fab′ and F(ab′)2, Fd, Fvs, single-chain Fvs (scFv), single-chain antibodies, disulfide-linked Fvs (sdFv), fragments comprising either a VL or VH domain, fragments produced by a Fab expression library, and anti-idiotypic (anti-Id) antibodies. ScFv molecules are known in the art and are described, e.g., in U.S. Pat. No. 5,892,019 Immunoglobulin or antibody molecules of the invention can be of any type (e.g., IgG, IgE, IgM, IgD, IgA, and IgY), class (e.g., IgG1, IgG2, IgG3, IgG4, IgA1 and IgA2) or subclass of immunoglobulin molecule.

Antigen-binding molecules, e.g., antibodies, or antigen-binding fragments, variants, or derivatives thereof may be described or specified in terms of the epitope(s) or portion(s) of an antigen, e.g., a target polypeptide that they recognize or specifically bind. The portion of a target polypeptide which specifically interacts with the antigen binding domain of an antibody is an “epitope,” or an “antigenic determinant.” A target polypeptide may comprise a single epitope, but typically comprises at least two epitopes, and can include any number of epitopes, depending on the size, conformation, and type of antigen. Furthermore, it should be noted that an “epitope” on a target polypeptide may be or include non-polypeptide elements, e.g., an epitope may include a carbohydrate side chain.

The antibodies can be polyclonal or monoclonal.

Polyclonal antibodies can be prepared by any known method. Polyclonal antibodies are raised by immunizing an animal (e.g. a rabbit, rat, mouse, donkey, and the like) by multiple subcutaneous or intraperitoneal injections of the relevant antigen (a purified peptide fragment, full-length recombinant protein, fusion protein, and the like) optionally conjugated to keyhole limpet hemocyanin (KLH), serum albumin, and the like, diluted in sterile saline and combined with an adjuvant (e.g., Complete or Incomplete Freund's Adjuvant) to form a stable emulsion. The polyclonal antibody is then recovered from blood, ascites and the like, of an animal so immunized. Collected blood is clotted, and the serum decanted, clarified by centrifugation, and assayed for antibody titer. The polyclonal antibodies can be purified from serum or ascites according to standard methods in the art including affinity chromatography, ion-exchange chromatography, gel electrophoresis, dialysis, and the like.

Monoclonal antibodies can be prepared using hybridoma methods, such as those described by Kohler and Milstein (1975) Nature 256:495, which is hereby incorporated by reference in its entirety. Using the hybridoma method, a mouse, hamster, or other appropriate host animal, is immunized as described above to elicit the production by lymphocytes of antibodies that will specifically bind to an immunizing antigen. Lymphocytes can also be immunized in vitro. Following immunization, the lymphocytes are isolated and fused with a suitable myeloma cell line using, for example, polyethylene glycol, to form hybridoma cells that can then be selected away from unfused lymphocytes and myeloma cells. Hybridomas that produce monoclonal antibodies directed specifically against a chosen antigen as determined by immunoprecipitation, immunoblotting, or by an in vitro binding assay (e.g., radioimmunoassay (RIA) and enzyme-linked immunosorbent assay (ELISA)) can then be propagated either in vitro culture using standard methods (Goding, Monoclonal Antibodies: Principles and Practice, Academic Press, 1986, which is hereby incorporated by reference in its entirety) or in vivo as ascites tumors in an animal. The monoclonal antibodies can then be purified from the culture medium or ascites fluid as described for polyclonal antibodies above.

Alternatively monoclonal antibodies can also be made using recombinant DNA methods as described in U.S. Pat. No. 4,816,567, which is hereby incorporated by reference in its entirety. The polynucleotides encoding a monoclonal antibody are isolated from mature B-cells or hybridoma cell, such as by RT-PCR using oligonucleotide primers that specifically amplify the genes encoding the heavy and light chains of the antibody, and their sequence is determined using conventional procedures. The isolated polynucleotides encoding the heavy and light chains are then cloned into suitable expression vectors, which when transfected into host cells such as E. coli cells, simian COS cells, Chinese hamster ovary (CHO) cells, or myeloma cells that do not otherwise produce immunoglobulin protein, monoclonal antibodies are generated by the host cells. Also, recombinant monoclonal antibodies or fragments thereof of the desired species can be isolated from phage display libraries expressing CDRs of the desired species as described (McCafferty et al. (1990) Nature 348:552-554; Clackson et al. (1991) Nature 352:624-628; and Marks et al. (1991) J. Mol. Biol. 222:581-597, each of which is hereby incorporated by reference in its entirety).

The polynucleotides encoding a monoclonal antibody can further be modified in a number of different manners using recombinant DNA technology to generate alternative antibodies. In some embodiments, the constant domains of the light and heavy chains of, for example, a mouse monoclonal antibody can be substituted 1) for those regions of, for example, a human antibody to generate a chimeric antibody or 2) for a non-immunoglobulin polypeptide to generate a fusion antibody. In some embodiments, the constant regions are truncated or removed to generate the desired antibody fragment of a monoclonal antibody. Site-directed or high-density mutagenesis of the variable region can be used to optimize specificity, affinity, and the like, of a monoclonal antibody.

Thus, in embodiments, the antibodies are humanized antibodies. In embodiments, the antibodies are chimeric antibodies.

Human antibodies can be directly prepared using various techniques known in the art. Immortalized human B lymphocytes immunized in vitro or isolated from an immunized individual that produce an antibody directed against a target antigen can be generated (See, e.g., Cole et al., Monoclonal Antibodies and Cancer Therapy, p. 77 (Alan R. Liss 1985); Boemer et al. (1991) J. Immunol. 147:86-95; and U.S. Pat. No. 5,750,373, each of which is hereby incorporated by reference in its entirety). Also, the human antibody can be selected from a phage library, where that phage library expresses human antibodies, as described, for example, in Vaughan et al. (1996) Nat. Biotech. 14:309-314, Sheets et al. (1998) Proc. Natl. Acad. Sci. 95:6157-6162, Hoogenboom and Winter (1991) J. Mol. Biol. 227:381, and Marks et al. (1991) J. Mol. Biol. 222:581, each of which is hereby incorporated by reference in its entirety. Techniques for the generation and use of antibody phage libraries are also described in U.S. Pat. Nos. 5,969,108, 6,172,197, 5,885,793, 6,521,404; 6,544,731; 6,555,313; 6,582,915; 6,593,081; 6,300,064; 6,653,068; 6,706,484; and 7,264,963; and Rothe et al. (2007) J. Mol. Bio. 376:1182-1200, each of which is incorporated by reference in its entirety. Affinity maturation strategies, such as chain shuffling (Marks et al. (1992) Bio/Technology 10:779-783, incorporated by reference in its entirety), are known in the art and may be employed to generate high affinity human antibodies.

Humanized antibodies can also be made in transgenic mice containing human immunoglobulin loci that are capable upon immunization of producing the full repertoire of human antibodies in the absence of endogenous immunoglobulin production. This approach is described in U.S. Pat. Nos. 5,545,807; 5,545,806; 5,569,825; 5,625,126; 5,633,425; and 5,661,016, each of which is hereby incorporated by reference in its entirety.

This invention also encompasses bispecific antibodies. Bispecific antibodies are antibodies that are capable of specifically recognizing and binding at least two different epitopes. The different epitopes can either be within the same molecule (e.g. the polynucleotide or polypeptide) or on different molecules such that both. Bispecific antibodies can be intact antibodies or antibody fragments.

It can further be desirable, especially in the case of antibody fragments, to modify an antibody in order to increase its serum half-life. This can be achieved, for example, by incorporation of a salvage receptor binding epitope into the antibody fragment by mutation of the appropriate region in the antibody fragment or by incorporating the epitope into a peptide tag that is then fused to the antibody fragment at either end or in the middle (e.g., by DNA or peptide synthesis).

Heteroconjugate antibodies are also within the scope of the present invention. Heteroconjugate antibodies are composed of two covalently joined antibodies. Such antibodies have, for example, been proposed to target immune cells to unwanted cells (U.S. Pat. No. 4,676,980, which is hereby incorporated by reference in its entirety). It is contemplated that the antibodies can be prepared in vitro using known methods in synthetic protein chemistry, including those involving crosslinking agents. For example, immunotoxins can be constructed using a disulfide exchange reaction or by forming a thioether bond. Examples of suitable reagents for this purpose include iminothiolate and methyl-4-mercaptobutyrimidate.

The present invention further embraces variants and equivalents which are substantially homologous to the chimeric, humanized and human antibodies, or antibody fragments thereof, set forth herein. These can contain, for example, conservative substitution mutations, e.g., the substitution of one or more amino acids by similar amino acids. For example, conservative substitution refers to the substitution of an amino acid with another within the same general class such as, for example, one acidic amino acid with another acidic amino acid, one basic amino acid with another basic amino acid or one neutral amino acid by another neutral amino acid. What is intended by a conservative amino acid substitution is well known in the art.

In aspects, the agent comprises a small molecule compound. In embodiments, the small molecule compound is an activator of glucocerebrosidase activity. See, e.g., International Patent Publication No. WO 2013/148333. In some embodiments, “small molecules” are molecules having low molecular weights (MW) that are capable of binding to a protein of interest thereby altering the function of the protein. In some embodiments, the MW of a small molecule is no more than 1,000. Methods for screening small molecules capable of altering protein function are known in the art. For example, a miniaturized arrayed assay for detecting small molecule-protein interactions in cells is discussed by You et al. (1997) Chem. Biol. 4:961-968.

In embodiments, the agent is a chaperone. As used herein, the term “chaperone” refers to a molecule, such as a small molecule, polypeptide, nucleic acid, and the like that specifically binds to a protein and has one or more of the following effects: restoring or enhancing at least partial wild-type function and/or activity of the protein; enhancing the formation of a stable molecular conformation of the protein; inducing trafficking of the protein from the ER to another cellular location, e.g., a native cellular location, thereby preventing ER-associated degradation of the protein; and/or preventing aggregation of misfolded proteins. In related embodiments, the chaperone restoring or enhancing at least partial wild-type function and/or activity of the protein. See, e.g., Patnaik et al. (2012) J. Med. Chem. 55:5734-5748. In other embodiments, the chaperone increases the residual activity of a cell (e.g., cell from a mammal suffering from a proteinopathy, a synucleinopathy, a tauopathy, or the like), optionally in combination with an agent that increases the activity of GBA1 (e.g., an agent described herein, including but not limited to, a GBA1 or equivalent thereof or a nucleic acid encoding a GBA1 or equivalent thereof). See, e.g., International Patent Publication No. WO 2012/177997; and Chang et al. (2006) FEBS J. 273:4082-4092.

In aspects, the invention involves administering at least two agents (e.g., combination therapy comprising administration of an agent that increases GBA1 activity in combination with another agent).

In some embodiments, an agent described herein is administered in combination with another therapeutic agent that is beneficial in treating a symptom associated with a proteinopathy, a synucleinopathy, a tauopathy, or the like). In embodiments, the agent described herein is a nucleic acid (e.g., a nucleic acid encoding a GBA1 or equivalent thereof). In embodiments, the agent described herein is a polypeptide (e.g., GBA1 or equivalent thereof). In embodiments, the agent described herein is a small molecule (e.g., activator of GBA1). In embodiments, the agent described herein is an antibody or fragment thereof (e.g., antibody or fragment thereof that specifically binds to GBA1). In embodiments, the agent described herein is a chaperone (e.g., chaperone of GBA1).

In some embodiments, the invention involves administering at least two of the agents described herein.

The phrase “combination therapy” embraces the administration of an agent that increases the activity of GBA1 and a second therapeutic agent as part of a specific treatment regimen intended to provide a beneficial effect from the co-action of these therapeutic agents. The beneficial effect of the combination includes, but is not limited to, pharmacokinetic or pharmacodynamic co-action resulting from the combination of therapeutic agents. Administration of these therapeutic agents in combination typically is carried out over a defined time period (usually minutes, hours, days, or weeks depending upon the combination selected). “Combination therapy” generally is not intended to encompass the administration of two or more of these therapeutic agents as part of separate monotherapy regimens that incidentally and arbitrarily result in the combinations of the present invention. “Combination therapy” is intended to embrace administration of these therapeutic agents in a sequential manner, that is, wherein each therapeutic agent is administered at a different time, as well as administration of these therapeutic agents, or at least two of the therapeutic agents, in a substantially simultaneous manner. Substantially simultaneous administration can be accomplished, for example, by administering to the subject a single capsule having a fixed ratio of each therapeutic agent or in multiple, single capsules for each of the therapeutic agents. Sequential or substantially simultaneous administration of each therapeutic agent can be effected by any appropriate route including, but not limited to, oral routes, intravenous routes, intramuscular routes, and direct absorption through mucous membrane tissues (e.g., nasal, mouth, vaginal, and rectal). The therapeutic agents can be administered by the same route or by different routes. For example, one component of a particular combination may be administered by intravenous injection while the other component(s) of the combination may be administered orally. The components may be administered in any therapeutically effective sequence. The phrase “combination” embraces groups of compounds or non-drug therapies useful as part of a combination therapy.

In any of the above aspects and embodiments, the agent can further contain a detectable moiety. Detectable moieties are well known in the art and can be detected by spectroscopic, photochemical, biochemical, immunochemical, physical, or chemical means. Exemplary moieties include, but are not limited to, enzymes, fluorescent molecules, particle labels, electron-dense reagents, radiolabels, biotin, digoxigenin, or a hapten or a protein that has been made detectable.

In any of the above aspects and embodiments, the agent can contain an additional chemical and/or biological moiety not normally part of the agent. Those derivatized moieties can improve delivery, solubility, biological half-life, absorption of the agent, and the like. The moieties can also reduce or eliminate any desirable side effects of the agent and the like. An overview for those moieties can be found in Remington's Pharmaceutical Sciences (20th ed., Mack Publishing Co. 2000) (see also Pathan et al. (2009) Recent Patents on Drug Delivery & Formulation 3:71-89, which is hereby incorporated by reference in its entirety).

The agent can be covalently or non-covalently linked to a moiety. In embodiments, the agent is covalently linked to the moiety. In related embodiments, the covalent linkage of the moiety is N-terminal to the polynucleotide/polypeptide. In related embodiments, the covalent linkage of the moiety is C-terminal to the polynucleotide/polypeptide.

In any instance of the above embodiments, the agent can be one that increases the glucocerebrosidase activity over baseline levels in the mammal. In certain embodiments, the glucocerebrosidase activity is increased by at least about 1.5 fold, about 2.0 fold, about 2.5 fold, about 3 fold, about 3.5 fold, about 4.0 fold, about 4.5 fold, about 5 fold, or more over baseline levels in the mammal. In certain embodiments, the glucocerebrosidase activity is increased in the neuron by at least about 1.5 fold, about 2.0 fold, about 2.5 fold, about 3 fold, about 3.5 fold, about 4.0 fold, about 4.5 fold, about 5 fold, or more over baseline levels. Baseline levels of glucocerebrosidase activity can be readily determined by methods known in the art and described herein. In some instances, the baseline level is the level that is exhibited, on average, by individuals without a proteinopathy or without GBA1 mutations.

Another aspect relates to a method for reducing α-synuclein in a mammal with a proteinopathy comprising administering a therapeutically effective amount of an agent that increases glucocerebrosidase activity. The α-synuclein can be found in different parts of the cell such as in the membrane, soluble in the cytosol, and insoluble in the cytosol. In certain embodiments, the methods described herein are effective in reducing a specific fraction of α-synuclein. In one embodiment, cytosolic soluble α-synuclein is reduced. In another embodiment, the membrane-associated α-synuclein is reduced. In embodiments, α-synuclein is reduced by at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or about 100%. In one embodiment, the α-synuclein is reduced to a level not significantly different than a mammal without a proteinopathy that is characterized by an increase in α-synuclein.

Another aspect relates to a method for reducing tau in a mammal with a proteinopathy comprising administering a therapeutically effective amount of an agent that increases glucocerebrosidase activity. Tau can be found in different parts of the cell such as in the membrane, soluble in the cytosol, and insoluble in the cytosol. In certain embodiments, the methods described herein are effective in reducing a specific fraction of tau. In one embodiment, cytosolic soluble tau is reduced. In another embodiment, the membrane-associated tau is reduced. In embodiments, tau is reduced by at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or about 100%. In one embodiment, the tau is reduced to a level not significantly different than a mammal without a proteinopathy that is characterized by an increase in tau.

Another aspect relates to a method for reducing toxic lipids (e.g., glucosylsphingosine) in a mammal with a proteinopathy comprising administering a therapeutically effective amount of an agent that increases glucocerebrosidase activity. In one embodiment, the toxic lipid is glucosylsphingosine. In further embodiments, the glucosylsphingosine is reduced by at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or about 100%. In one embodiment, the glucosylsphingosine is reduced to a level not significantly different than a mammal without a proteinopathy that is characterized by an increase in glucosylsphingosine.

Another aspect relates to a method for inhibiting the accumulation of protein aggregates in a mammal with a proteinopathy comprising administering a therapeutically effective amount of an agent that increases glucocerebrosidase activity. In a related embodiment, the protein aggregate is selected from the group consisting of ubiquitin, tau, and α-synuclein.

### Compositions and Kits

Also provided by this invention is a composition or kit comprising any one or more of the agents described herein, useful for increasing glucocerebrosidase activity in a mammal in need thereof. These compositions can and kits be used therapeutically as described herein and can be used in combination with other known therapies for proteinopathies. For example, common treatments for proteinopathies include Levodopa, dopamine agonists, MAO-B inhibitors, amantadine, anticholinergics, surgery, rehabilitation, and diet management. Common therapies for Alzheimer's include, for example, acetylcholinesterase inhibitors such as tacrine, rivastigmine, galantamine, donepezil, memantine. Further therapies for proteinopathies include psychosocial interventions, behavioural interventions, reminiscence therapy, validation therapy, supportive psychotherapy, sensory integration, cognitive retraining, rehabilitation, speech therapy, and the like.

A “pharmaceutical composition” can include an agent and another carrier, e.g., compound or composition, inert or active, such as a detectable agent, label, adjuvant, diluent, binder, stabilizer, buffers, salts, lipophilic solvents, preservative, adjuvant or the like. Carriers also include pharmaceutical excipients and additives, for example, proteins, peptides, amino acids, lipids, and carbohydrates (e.g., sugars, including monosaccharides, di-, tri-, tetra-, and oligosaccharides; derivatized sugars such as alditols, aldonic acids, esterified sugars and the like; and polysaccharides or sugar polymers), which can be present singly or in combination, comprising alone or in combination 1-99.99% by weight or volume. Exemplary protein excipients include serum albumin such as human serum albumin (HSA), recombinant human albumin (rHA), gelatin, casein, and the like. Representative amino acid/antibody components, which can also function in a buffering capacity, include alanine, glycine, arginine, betaine, histidine, glutamic acid, aspartic acid, cysteine, lysine, leucine, isoleucine, valine, methionine, phenylalanine, aspartame, and the like. Carbohydrate excipients are also intended within the scope of this invention, examples of which include but are not limited to monosaccharides such as fructose, maltose, galactose, glucose, D-mannose, sorbose, and the like; disaccharides, such as lactose, sucrose, trehalose, cellobiose, and the like; polysaccharides, such as raffinose, melezitose, maltodextrins, dextrans, starches, and the like; and alditols, such as mannitol, xylitol, maltitol, lactitol, xylitol sorbitol (glucitol) and myoinositol.

The term carrier further includes a buffer or a pH adjusting agent; typically, the buffer is a salt prepared from an organic acid or base. Representative buffers include organic acid salts such as salts of citric acid, ascorbic acid, gluconic acid, carbonic acid, tartaric acid, succinic acid, acetic acid, or phthalic acid; Tris, tromethamine hydrochloride, or phosphate buffers. Additional carriers include polymeric excipients/additives such as polyvinylpyrrolidones, ficolls (a polymeric sugar), dextrates (e.g., cyclodextrins, such as 2-hydroxypropyl-.quadrature.-cyclodextrin), polyethylene glycols, flavoring agents, antimicrobial agents, sweeteners, antioxidants, antistatic agents, surfactants (e.g., polysorbates such as “TWEEN 20” and “TWEEN 80”), lipids (e.g., phospholipids, fatty acids), steroids (e.g., cholesterol), and chelating agents (e.g., EDTA).

As used herein, the term “pharmaceutically acceptable carrier” encompasses any of the standard pharmaceutical carriers, such as a phosphate buffered saline solution, water, and emulsions, such as an oil/water or water/oil emulsion, and various types of wetting agents. The compositions also can include stabilizers and preservatives and any of the above noted carriers with the additional provisio that they be acceptable for use in vivo. For examples of carriers, stabilizers and adjuvants, see Remington's Pharmaceutical Sciences (20th ed., Mack Publishing Co. 2000) and the Physician's Desk Reference (52^{nd} ed., Medical Economics 1998).

Generally, the agents and compositions described herein are administered in an effective amount or quantity sufficient to augment glucocerebrosidase activity in a subject. Typically, the dose can be adjusted within this range based on, e.g., age, physical condition, body weight, sex, diet, time of administration, and other clinical factors. Determination of an effective amount is well within the capability of those skilled in the art.

Methods of delivery of the compositions described herein include but are not limited to oral, non-oral (e.g., topically, transdermally, by inhalation, or by injection). Such modes of administration and the methods for preparing an appropriate pharmaceutical composition for use therein are described in Gibaldi's Drug Delivery Systems in Pharmaceutical Care (1st ed., American Society of Health-System Pharmacists 2007), which is hereby incorporated by reference.

In embodiments, the pharmaceutical compositions are administered orally in a solid form.

Pharmaceutical compositions suitable for oral administration can be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a compound(s) described herein, a derivative thereof, or a pharmaceutically acceptable salt or prodrug thereof as the active ingredient(s). The active ingredient can also be administered as a bolus, electuary, or paste.

In solid dosage forms for oral administration (e.g., capsules, tablets, pills, dragees, powders, granules and the like), the active ingredient is mixed with one or more pharmaceutically acceptable carriers, excipients, or diluents, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, acetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof; and (10) coloring agents. In the case of capsules, tablets, and pills, the pharmaceutical compositions can also comprise buffering agents. Solid compositions of a similar type can also be prepared using fillers in soft and hard-filled gelatin capsules, and excipients such as lactose or milk sugars, as well as high molecular weight polyethylene glycols and the like.

A tablet can be made by compression or molding, optionally with one or more accessory ingredients. Compressed tablets can be prepared using binders (for example, gelatin or hydroxypropylmethyl cellulose), lubricants, inert diluents, preservatives, disintegrants (for example, sodium starch glycolate or cross-linked sodium carboxymethyl cellulose), surface-actives, and/or dispersing agents. Molded tablets can be made by molding in a suitable machine a mixture of the powdered active ingredient moistened with an inert liquid diluent. The tablets and other solid dosage forms, such as dragees, capsules, pills, and granules, can optionally be scored or prepared with coatings and shells, such as enteric coatings and other coatings well known in the art.

The pharmaceutical compositions can also be formulated so as to provide slow, extended, or controlled release of the active ingredient therein using, for example, hydroxypropylmethyl cellulose in varying proportions to provide the desired release profile, other polymer matrices, liposomes and/or microspheres. The pharmaceutical compositions can also optionally contain opacifying agents and may be of a composition that releases the active ingredient(s) only, or preferentially, in a certain portion of the gastrointestinal tract, optionally, in a delayed manner. Examples of embedding compositions include polymeric substances and waxes. The active ingredient can also be in micro-encapsulated form, if appropriate, with one or more pharmaceutically acceptable carriers, excipients, or diluents well known in the art (see, e.g., Remington's).

In embodiments, the pharmaceutical compositions are administered orally in a liquid form. Liquid dosage forms for oral administration of an active ingredient include pharmaceutically acceptable emulsions, microemulsions, solutions, suspensions, syrups and elixirs. In addition to the active ingredient, the liquid dosage forms can contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (e.g., cottonseed, groundnut, corn, germ, olive, castor and sesame oils), glycerol, tetrahydrofuryl alcohol, polyethylene glycols and fatty acid esters of sorbitan, and mixtures thereof. In addition to inert diluents, the liquid pharmaceutical compositions can include adjuvants such as wetting agents, emulsifying and suspending agents, sweetening, flavoring, coloring, perfuming and preservative agents, and the like.

Suspensions, in addition to the active ingredient(s) can contain suspending agents such as, but not limited to, ethoxylated isostearyl alcohols, polyoxyethylene sorbitol and sorbitan esters, microcrystalline cellulose, aluminum metahydroxide, bentonite, agar-agar and tragacanth, and mixtures thereof.

In embodiments, the pharmaceutical compositions are administered by non-oral means such as by topical application, transdermal application, injection, and the like. In related embodiments, the pharmaceutical compositions are administered parenterally by injection, infusion, or implantation (e.g., intravenous, intramuscular, intra-arterial, subcutaneous, and the like).

In aspects, it may be desirable to administer the pharmaceutical compositions and/or cells of the disclosure directly to the CNS. Accordingly, in certain embodiments, the compositions are administered directly to the CNS so as to avoid the blood brain barrier. In some embodiments, the composition can be administered via direct spinal cord injection. In embodiments, the composition is administered by intrathecal injection. In some embodiments, the composition is administered via intracerebroventricular injection. In embodiments, the composition is administered into a cerebral lateral ventricle. In embodiments, the composition is administered into both cerebral lateral ventricles. In additional embodiments, the composition is administered via intrahippocampal injection.

The compositions may be administered in one injection or in multiple injections. In other embodiments, the composition is administered to more than one location (e.g., two sites to the CNS).

Compositions for parenteral use can be presented in unit dosage forms, e.g., in ampoules or in vials containing several doses, and in which a suitable preservative can be added. Such compositions can be in form of a solution, a suspension, an emulsion, an infusion device, a delivery device for implantation, or it can be presented as a dry powder to be reconstituted with water or another suitable vehicle before use. One or more co-vehicles, such as ethanol, can also be employed. Apart from the active ingredient(s), the compositions can contain suitable parenterally acceptable carriers and/or excipients or the active ingredient(s) can be incorporated into microspheres, microcapsules, nanoparticles, liposomes, or the like for controlled release. Furthermore, the compositions can also contain suspending, solubilising, stabilising, pH-adjusting agents, and/or dispersing agents.

The pharmaceutical compositions can be in the form of sterile injections. The pharmaceutical compositions can be sterilized by, for example, filtration through a bacteria-retaining filter, or by incorporating sterilizing agents in the form of sterile solid compositions which can be dissolved in sterile water, or some other sterile injectable medium immediately before use. To prepare such a composition, the active ingredient is dissolved or suspended in a parenterally acceptable liquid vehicle. Exemplary vehicles and solvents include, but are not limited to, water, water adjusted to a suitable pH by addition of an appropriate amount of hydrochloric acid, sodium hydroxide or a suitable buffer, 1,3-butanediol, Ringer's solution and isotonic sodium chloride solution. The pharmaceutical composition can also contain one or more preservatives, for example, methyl, ethyl or n-propyl p-hydroxybenzoate. To improve solubility, a dissolution enhancing or solubilising agent can be added or the solvent can contain 10-60% w/w of propylene glycol or the like.

The pharmaceutical compositions can contain one or more pharmaceutically acceptable sterile isotonic aqueous or nonaqueous solutions, dispersions, suspensions or emulsions, or sterile powders, which can be reconstituted into sterile injectable solutions or dispersions just prior to use. Such pharmaceutical compositions can contain antioxidants; buffers; bacteriostats; solutes, which render the formulation isotonic with the blood of the intended recipient; suspending agents; thickening agents; preservatives; and the like.

Examples of suitable aqueous and nonaqueous carriers, which can be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants. In some embodiments, in order to prolong the effect of an active ingredient, it is desirable to slow the absorption of the compound from subcutaneous or intramuscular injection. This can be accomplished by the use of a liquid suspension of crystalline or amorphous material having poor water solubility. The rate of absorption of the active ingredient then depends upon its rate of dissolution which, in turn, can depend upon crystal size and crystalline form. Alternatively, delayed absorption of a parenterally-administered active ingredient is accomplished by dissolving or suspending the compound in an oil vehicle. In addition, prolonged absorption of the injectable pharmaceutical form can be brought about by the inclusion of agents that delay absorption such as aluminum monostearate and gelatin.

Controlled release parenteral compositions can be in form of aqueous suspensions, microspheres, microcapsules, magnetic microspheres, oil solutions, oil suspensions, emulsions, or the active ingredient can be incorporated in biocompatible carrier(s), liposomes, nanoparticles, implants or infusion devices.

Materials for use in the preparation of microspheres and/or microcapsules include, but are not limited to, biodegradable/bioerodible polymers such as polyglactin, poly-(isobutyl cyanoacrylate), poly(2-hydroxyethyl-L-glutamine) and poly(lactic acid).

Biocompatible carriers which can be used when formulating a controlled release parenteral formulation include carbohydrates such as dextrans, proteins such as albumin, lipoproteins or antibodies.

Materials for use in implants can be non-biodegradable, e.g., polydimethylsiloxane, or biodegradable such as, e.g., poly(caprolactone), poly(lactic acid), poly(glycolic acid) or poly(ortho esters).

Having been generally described herein, the follow examples are provided to further illustrate this invention.

### EXAMPLES

### Augmenting Glucocerebrosidase Activity in the CNS as a Therapeutic Strategy for Gaucher-Related Tauopathies and Other Proteinopathies

Mutations of GBA1, the gene encoding glucocerebrosidase, represent a common genetic risk factor for developing the synucleinopathies Parkinson's disease (PD) and dementia with Lewy bodies (DLB). PD patients with or without GBA1 mutations also exhibit lower enzymatic levels of glucocerebrosidase in the central nervous system (CNS), suggesting a possible link between the enzyme and the development of the disease. This example describes the augmentation of glucocerebrosidase activity in the CNS of a mouse model of Gaucher-related synucleinopathy (Gba1^{D409V/D409V}) and a transgenic mouse overexpressing A53T α-synuclein. Example 1 demonstrates that adeno-associated virus-mediated expression of glucocerebrosidase in the CNS of symptomatic Gba1^{D409V/D409V} mice completely corrected the aberrant accumulation of the toxic lipid glucosylsphingosine and reduced the levels of ubiquitin, tau and proteinase-K-resistant α-synuclein aggregates. Importantly, hippocampal expression of glucocerebrosidase in Gba1^{D409V/D409V} mice (starting at 4 or 12 months old) also reversed their cognitive impairment when examined using the novel object recognition test. Overexpression of glucocerebrosidase in the CNS of A53T α-synuclein mice reduced the levels of soluble α-synuclein, suggesting that this glycosidase can modulate the development of α-synucleinopathies. Hence, increasing glucocerebrosidase activity in the CNS represents a potential therapeutic strategy for GBA1-related and non-GBA1-associated tauopathies.

Mutations in the gene for glucocerebrosidase (GBA1) reportedly present the highest genetic risk factor for developing synucleinopathies such as Parkinson's disease (PD) and dementia with Lewy bodies (DLB) (See, e.g., Aharon-Peretz J et al. (2004) N Engl J Med 351:1972-1977; Sidransky E et al. (2009) N Engl J Med 361:1651-1661; Velayati A et al. (2010) Curr Neurol Neurosci Rep 10:190-198; Clark L N et al. (2007) Neurology 69:1270-1277; Mata I F et al. (2008) Arch Neurol 65:379-382; Bultron G et al. (2010) J Inherit Metab Dis 33:167-173; Rosenbloom B et al. (2010) Blood Cells Mol Dis 46:95-102; and Duran R et al. (2012) Mol Genet Metab 4:495-497). The central nervous systems (CNS) of Gaucher patients and carriers who present with Parkinsonism and dementia harbor deposits of α-synuclein-positive Lewy bodies (LB) and Lewy neurites (LN) in hippocampal neurons and processes that are similar to those noted in patients with classical PD and DLB (See, for example, Spillantini M G et al. (1997) Nature 388:839-840; Spillantini M G et al. (1998) Proc Natl Acad Sci USA 95:6469-6473; Tayebi N et al. (2003) Mol Genet Metab 79:104-109 and Wong K et al. (2004) Mol Genet Metab 82:192-207. Aspects of these characteristics have also been noted in the CNS of several mouse models of neuropathic and non-neuropathic Gaucher disease (See, for example, Xu Y H et al. Mol Genet Metab (2010) 102:436-447; Cullen V et al. (2011) Ann Neurol 69:940-953; and Sardi S P et al. (2011) Proc Natl Acad Sci USA 108:12101-12106). Consequently, a causal relationship between the loss of glucocerebrosidase activity or the lysosomal accumulation of undegraded metabolites and the development of PD and DLB has been suggested. A more direct link between glucocerebrosidase activity and α-synuclein metabolism has been highlighted by studies of Gaucher cells and mice that showed that a reduction in glucocerebrosidase activity by pharmacological or genetic interventions resulted in increased levels of α-synuclein aggregates (See, for example, Cullen V et al. (2011) Ann Neurol 69:940-953; Sardi S P et al. (2011) Proc Natl Acad Sci USA 108:12101-12106; Manning-Bog A B et al. (2009) Neurotoxicology 30:1127-1132; and Mazzulli J R et al. (2011) Cell 146:37-52). Moreover, a decrease in glucocerebrosidase activity has been noted in CSF and brain samples from subjects with PD and DLB (regardless of whether they harbor mutations in GBA1), suggesting that a reduction in glucocerebrosidase activity may contribute to the development of synucleinopathies (see, for example, Balducci C et al. (2007) Mov Disord 22:1481-1484; Parnetti L et al. (2009) Neurobiol Dis 34:484-486; and Gegg M E et al. (2012) Annals of Neurology 72:455-63).

A role for glucocerebrosidase in the development of synucleinopathies is further supported by clinical observations of subjects with Gaucher-associated Parkinsonism. These individuals present with increased frequencies and severities of non-motor symptoms (e.g., cognitive impairment) that substantially erode their quality of life (see, for example, Brockmann K et al. (2011) Neurology 77:276-280; McNeill A et al. (2012) Mov Disord 27:526-532; and McNeill A et al. (2012) J Neurol Neurosurg Psychiatry 83:253-254). Individuals harboring mutations in GBA1 also have a higher incidence of dementia that is correlated with the presence of neocortical accumulation of aggregates of α-synuclein (see, e.g., Clark L N et al. (2009) Arch Neurol 66:578-583; and Neumann J et al. (2009) Brain 132:1783-1794). Indeed, mutations in GBA1 are now recognized as an independent risk factor for developing cognitive impairment in PD patients (see, e.g., Alcalay R N et al. (2012) Neurology 78:1434-1440). Another gene that has been shown to be associated with an increased risk for developing dementia in PD is MART (see, for example, Goris A et al. (2007) Ann Neurol 62:145-153). This gene encodes tau, a microtubule-associated protein that has a role in maintaining the proper organization and integrity of the cytoskeleton. Tau- and α-synuclein-associated pathologies are frequently found in tandem in patients with PD and LBD (see, for example, McKeith I G et al. (1996) Neurology 47:1113-1124; Duda J E et al. (2002) Acta Neuropathol 104:7-11; and Giasson B I et al. (2003) Science 300:636-640).

Mutations in GBA1 with resultant deficiency in glucocerebrosidase activity are the molecular basis of Gaucher disease, the most prevalent member of the family of lysosomal storage disorders (see, for example, Brady R O et al. (1966) J Clin Invest 45:1112-1115 and Sidransky (2004) Mol Genet Metab 83:6-15). The disease is characterized by the progressive accumulation of unmetabolized lipid substrates, primarily glucosylceramide, in the lysosomes. Subjects with Gaucher disease are presently managed by periodic administrations of a glycan-modified recombinant glucocerebrosidase (see, for example, Cox T M (2001) QJM 94:399-402 and Grabowski G A (2008) Lancet 372:1263-1271). However, the recombinant enzyme is unable to traverse the blood brain barrier in sufficient quantities to address the CNS manifestations of neuropathic Gaucher patients (see, for example, Grabowski G A (2008) Lancet 372:1263-1271 and Grabowski G A et al. (1998) Blood Rev 12:115-133). Strategies to augment glucocerebrosidase levels in the CNS have recently been the subject of intense investigation (see, for example, Cabrera-Salazar M A et al. (2010) Exp Neurol 225:436-444; Khanna R et al. (2010) FEBS J 277:1618-1638; Ashe K M et al. (2011) PLoS One 6:e21758; and Patnaik S et al. (2012) J Med Chem 55:5734-5748).

A mouse model of Gaucher-related synucleinopathy that exhibits progressive CNS accumulation of proteinase K-resistant α-synuclein/ubiquitin aggregates that are reminiscent of Lewy neurites has previously been described (Sardi S P et al. (2011) Proc. Natl. Acad. Sci. USA 108:12101-12106). These mice also display higher levels of the neurotoxin glucosylsphingosine (GlcSph) in their CNS and a demonstrable hippocampal memory deficit. This example characterizes the pathological features associated with this model of Gaucher-associated synucleinopathy to include the protein tau. Moreover, it was examined whether the aberrations can be moderated or reversed when glucocerebrosidase was administered into animals at a clinically relevant post-symptomatic stage. Finally, to further probe the relationship between glucocerebrosidase and α-synuclein, the capacity of the lysosomal hydrolase to affect α-synuclein levels in the A53T α-synuclein mouse was evaluated as described herein.

### Example 1

### The CNS of a Mouse Model of Gaucher Disease Exhibits Accumulation of Tau Aggregates

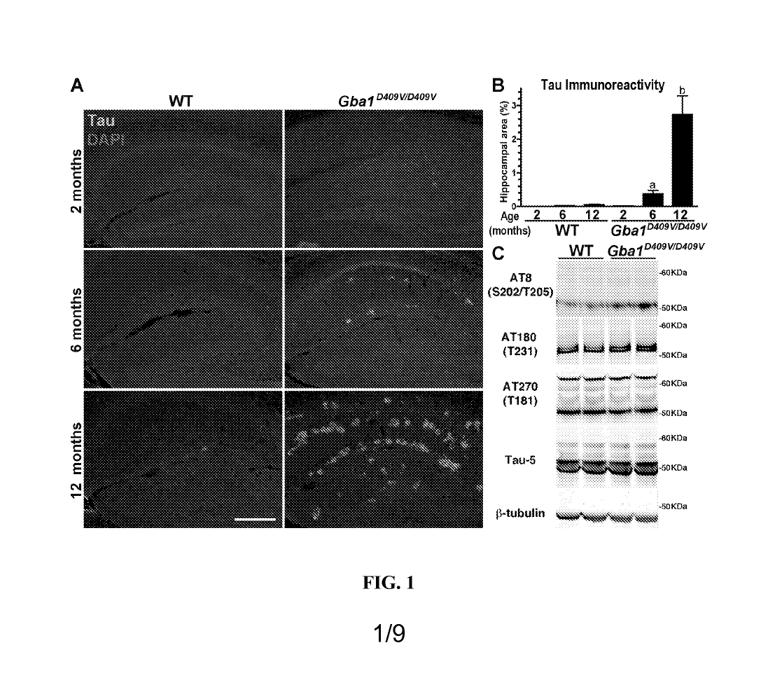

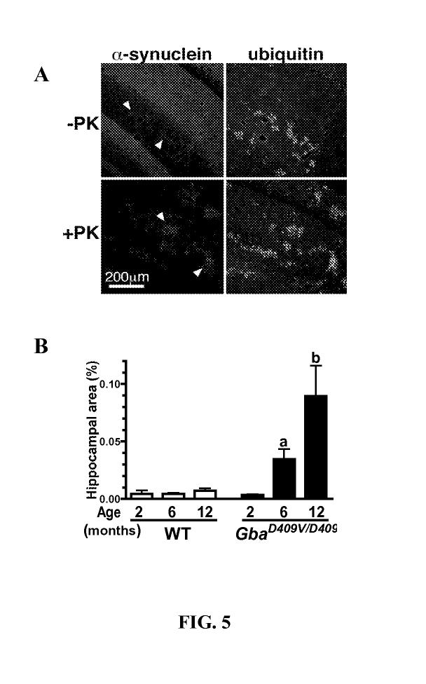

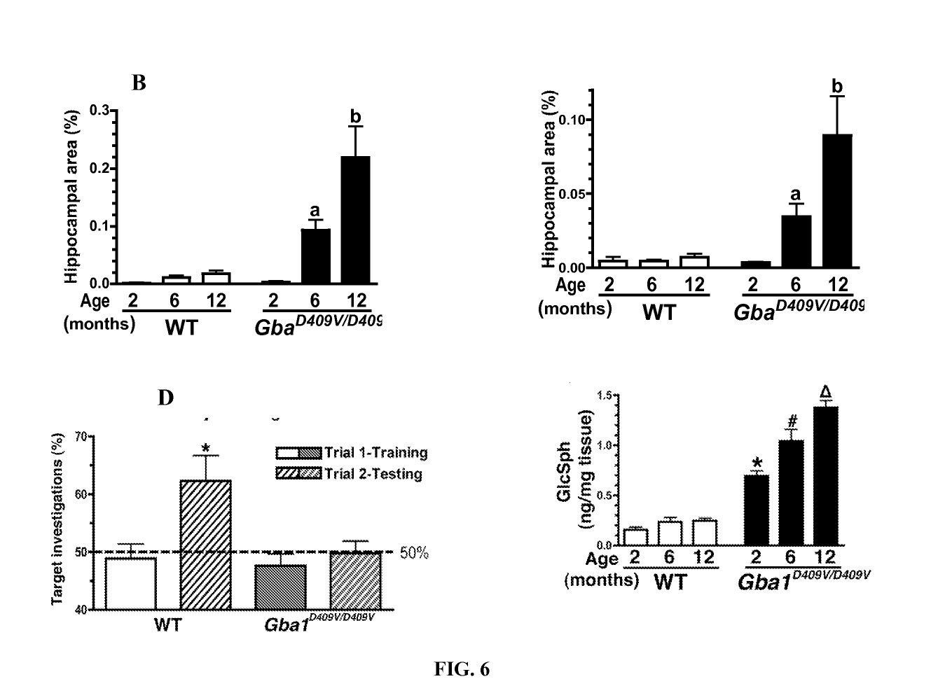

Accumulation of α-synuclein and tau inclusions with resultant dementia are the hallmarks of a number of neurodegenerative diseases, including PD and DLB (see, for example, McKeith I G et al. (1996) Neurology 47:1113-1124; Ishizawa T et al. (2003) J Neuropathol Exp Neurol 62:389-397; and Lee V M et al. (2004) Trends Neurosci 27:129-134). It was previously reported that a mouse model of Gaucher disease harboring a single point mutation in the murine Gba1 locus (Gba1^{D409V/D409V}) exhibits progressive and marked accumulation of α-synuclein/ubiquitin aggregates in the CNS and a measurable deficit in hippocampal memory (Sardi S P et al. (2011) Proc Natl Acad Sci USA 108:12101-12106) (see also FIGS. 5A and B and FIG. 6A-D). To determine if mutations in Gba1 with resultant loss of glucocerebrosidase activity also promote the accumulation of tau in the CNS, brain sections of 12-month-old Gba1^{D409V/D409V} mice were examined immunohistochemically using an antibody that specifically recognizes tau. Marked punctate staining was noted primarily in the hippocampal regions (FIG. 1A), although evidence of immunoreactivity was also observed in other brain areas, such as the cerebral cortex and the cerebellum. The onset and rate of accumulation of the tau aggregates in the brains of Gba1^{D409V/D409V} mice were also determined At 2 months of age, the extent of tau immunoreactivity in Gba1^{D409V/D409V} mice was not different from that noted in wild-type controls (FIGS. 1A and B). However, the level of tau staining in 6-month-old Gba1^{D409V/D409V} mice was significantly higher than that in the age-matched controls. Accumulation was progressive, with 12-month-old Gba1^{D409V/D409V} mice displaying higher amounts of tau aggregates (FIGS. 1A and B).

A common finding in neurodegenerative diseases is an increase in the presence of the hyperphosphorylated tau that comprises the neurofibrillary tangles (see, for example, Goedert M et al. (1995) Neurosci Lett 189:167-169; and Hanger D P et al. (2009) Trends Mol Med 15:112-119). These phosphorylated species can be detected using specific antibodies, such as AT270 (which recognizes tau phosphorylated at Thr181), AT8 (which recognizes tau phosphorylated at Ser202 and Thr205), and AT180 (which recognizes tau phosphorylated at Thr231). To probe the phosphorylation status of the tau aggregates in the CNS of Gba1^{D409V/D409V} mice, western blot analysis was performed on hippocampal lysates from 18-month-old mice. Staining the blots using an antibody (Tau-5) that recognizes all tau species revealed that the overall levels of the protein were not different between Gba1^{D409V/D409V} and wild type mice (FIG. 1C). No differences in the extent of staining between controls and age-matched Gba1^{D409V/D409V} mice were observed when the blots were probed using either AT180 or AT270 antibodies (FIG. 1C). However, AT8 staining, which detects phosphorylation on Ser202 and Thr205, was modestly but significantly increased in the lysates of Gba1^{D409V/D409V} mice (1.3±0.1 compared to wild-type, n=6, p<0.05, FIG. 1C). This observation of increased phosphorylation on Ser202 and Thr205, coupled with the progressive nature of the accumulation of the tau aggregates (in addition to α-synuclein), indicates that the CNS of Gba1^{D409V/D409V} mice recapitulate pathological features noted in subjects with PD and DLB.

### Example 2

### Administration of Glucocerebrosidase into the Hippocampus Reverses the Biochemical and Memory Aberrations of Post-Symptomatic Gba1^{D409V/D409V} Mice

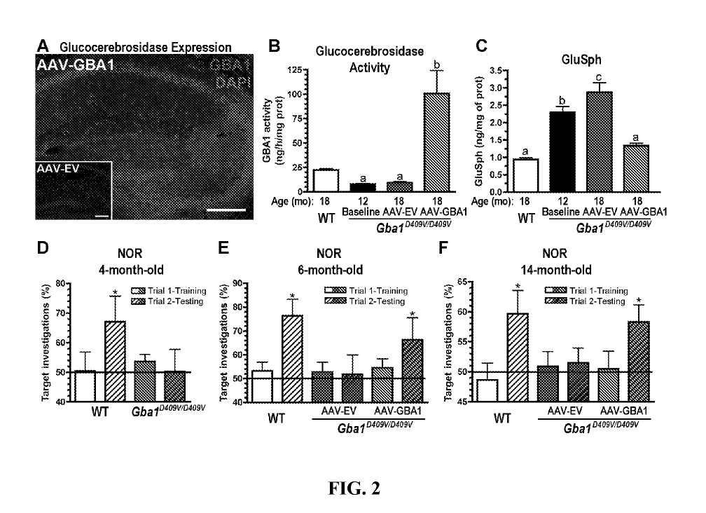

To determine if reconstitution of the CNS with recombinant glucocerebrosidase can correct the biochemical aberrations and memory deficits of symptomatic Gba1^{D409V/D409V} mice, a recombinant self-complementary adeno-associated viral vector (serotype 1) encoding human glucocerebrosidase (AAV-GBA1) was administered bilaterally into the hippocampi of early and late symptomatic mice (4- and 12-month-old, respectively). Immunohistochemical examination of the CNS of Gba1^{D409V/D409V} mice that were administered AAV-GBA1 at 12 months of age and then analyzed 6 months later revealed abundant and widespread hippocampal expression of glucocerebrosidase (FIG. 2A). Mice treated with a control virus that did not encode a transgene (AAV-EV) showed no staining (FIG. 2A, inset). The enzymatic activity in AAV-GBA1-treated (FIG. 2B, red bar) mice was determined to be approximately 10-fold higher than that at baseline (FIG. 2B, black bar) and that of Gba1^{D409V/D409V} mice administered AAV-EV (FIG. 2B, blue bar). A similar distribution of the enzyme was noted in the CNS of Gba1^{D409V/D409V} mice treated at 4 months of age and analyzed 6 months post-treatment (data not shown). Expression of glucocerebrosidase in the 12-month-old mice was associated with normalization of the hyper-elevated levels of brain glucosylsphingosine after 6 months (FIG. 2C, red bar). In contrast, Gba1^{D409V/D409V} mice treated with the control virus exhibited continued accumulation of the pro-inflammatory lipid over the same time interval (FIG. 2C, blue bar).

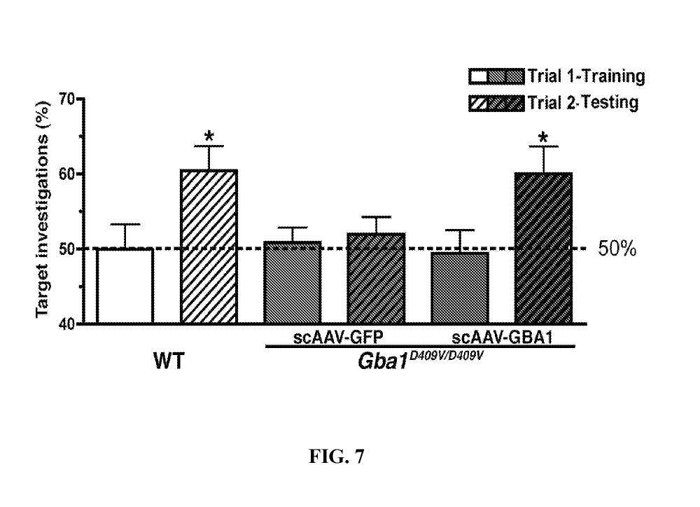

Hippocampal memory was evaluated using the novel object recognition test. Testing of 4-month-old Gba1^{D409V/D409V} mice prior to treatment confirmed that they exhibited impairments in novel object recollection (FIG. 2D). Treatment of these mice with AAV-GBA1 reversed memory deficits when the mice were tested 2 months later (at 6 months old; FIG. 2E, red bars, n=10, p<0.05). In contrast, Gba1^{D409V/D409V} mice treated with the control viral vector showed no discernible improvement (FIG. 2E, blue bars, n=9). A similar result was attained in a separate cohort of Gba1^{D409V/D409V} mice treated with AAV-GBA1 at 12 months of age (i.e., with higher levels of pre-existing pathology) and tested 2 months later (at 14 months old; FIG. 2F, red bars, n=12, p<0.05; AAV-EV, blue bars, n=12). Hence, augmenting glucocerebrosidase activity in the CNS of post-symptomatic Gba1^{D409V/D409V} mice corrected the pathological accumulation of glucosylsphingosine and, importantly, their memory impairments (see also FIG. 7 showing that GBA1 augmentation can also correct memory deficit in 2 month old Gba1^{D409V/D409V} mice).

### Example 3

### Administration of Glucocerebrosidase into the Hippocampus of Symptomatic Gba1^{D409V/D409V} Mice Reduces the Levels of Aggregated Proteins in the Brain

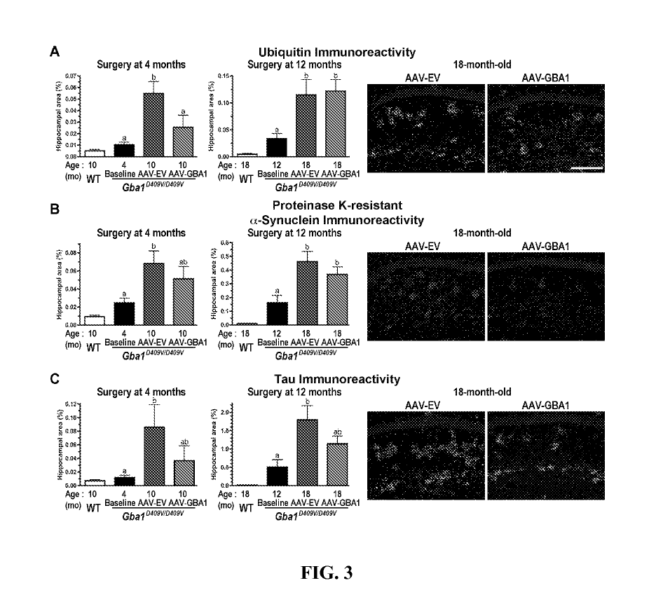

As Gba1^{D409V/D409V} mice exhibit reduced glucocerebrosidase activity and progressive accumulation of ubiquitin, α-synuclein and tau aggregates in the hippocampus, it was tested whether augmenting glucocerebrosidase levels in the brain would decrease the levels of these aberrant proteinaceous materials in post-symptomatic animals. The hippocampi of 4- and 12-month-old Gba1^{D409V/D409V} mice (the latter presented with greater accumulation of aggregates and pathology) were stereotaxically injected bilaterally with 2E11 DNase-resistant particles (drp) of AAV-GBA1 or AAV-EV. Analysis of brain tissues of Gba1^{D409V/D409V} mice at the start of the study (at 4 and 12 months of age) and at 6 months post-injection with the control AAV-EV vector showed accumulation of ubiquitin, α-synuclein and tau aggregates over this period (FIG. 3 A-C). In contrast, gene delivery of AAV-GBA1 into the 4-month-old Gba1^{D409V/D409V} mice led to reductions of hippocampal ubiquitin, proteinase K-resistant α-synuclein and tau aggregates (FIG. 3A-C). However, the reduction of ubiquitin, but not the reductions in α-synuclein or tau, reached statistical significance. CNS expression of glucocerebrosidase in the older (12-month-old) mice produced a similar, but more modest, effect than that noted in the younger cohort when assayed 6 months later (FIG. 3A-C). Delivery of glucocerebrosidase appeared to have slowed the rates of accumulation of tau and α-synuclein but had no effect on ubiquitin levels, suggesting the mechanisms for accumulation of these proteins may be different. It is possible that the higher levels of aggregates present in the older animals require a longer period or more glucocerebrosidase to be efficiently reduced. Nevertheless, the data suggest that augmenting glucocerebrosidase activity in the CNS can retard the extent of accumulation of pathologically misfolded protein aggregates in symptomatic Gba1^{D409V/D409V} mice.

### Example 4

### The CNS of Transgenic A53T α-Synuclein Mice are Associated with Lower Glucocerebrosidase Activities

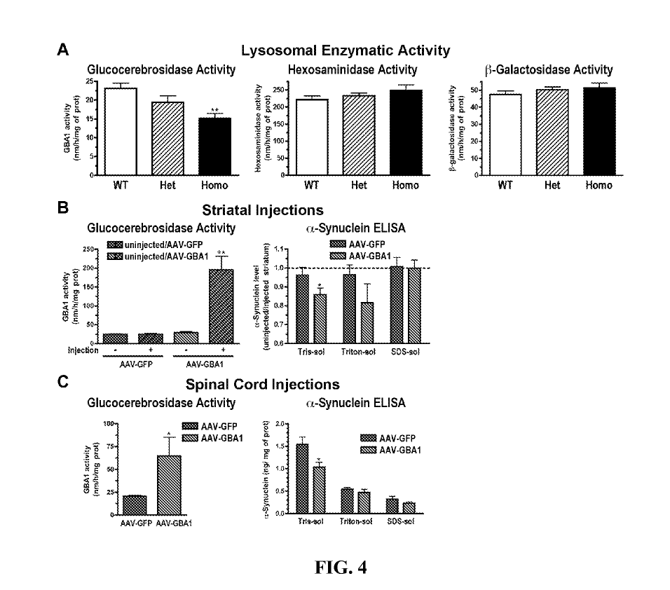

Analyses of CSF and brain samples of subjects with PD or DLB have shown that glucocerebrosidase activity is lower in affected than in unaffected individuals, suggesting a causal role of the lysosomal enzyme in the development of these synucleinopathies (see, for example, Balducci C et al. (2007) Mov Disord 22:1481-1484; Parnetti L et al. Neurobiol Dis (2009) 34:484-486; and Gegg M E et al. (2012) Annals of Neurology 72:455-63). Recent data has also suggested that α-synuclein has the capacity to inhibit lysosomal glucocerebrosidase activity (see, for example, Mazzulli J R et al. (2011) Cell 146:37-52 and Yap T L et al. (2011) J Biol Chem 286:28080-28088). To determine whether overexpression of α-synuclein negatively affects the activity of glucocerebrosidase, brain lysates from transgenic A53T α-synuclein mice (expressing mutant human α-synuclein bearing the A53T mutation) were studied. Similar to findings in PD patients without mutations in GBA1, A53T α-synuclein mice exhibited significantly lower lysosomal glucocerebrosidase activity than did wild type animals (FIG. 4A). This effect was dependent on the levels of α-synuclein, as the CNS of homozygous A53T α-synuclein mice showed greater reductions in enzymatic activity than their (Het) littermates who expressed lower levels of α-synuclein (FIG. 4A, hatched bars). This decrease was selectively associated with glucocerebrosidase, as the activities of other lysosomal enzymes (i.e., hexosaminidase and β-galactosidase) were unaffected (FIG. 4A). These results support the contention that high levels of α-synuclein can inhibit lysosomal glucocerebrosidase activity, since greater inhibition was correlated with higher levels of α-synuclein.

### Example 5

### AAV-Mediated Expression of Glucocerebrosidase in the CNS of Transgenic A53T α-Synuclein Mice Lowers α-Synuclein Levels

Earlier, it was noted that overexpression of glucocerebrosidase reduced the accumulation of α-synuclein aggregates in the CNS of symptomatic Gba1^{D409V/D409V} mice (FIG. 3B). To confirm the therapeutic potential of glucocerebrosidase in moderating the accumulation of α-synuclein, it was next tested whether this reduction could also be realized in A53T α-synuclein mice. The striata of 4-month-old heterozygous A53T α-synuclein mice were unilaterally injected with either AAV-GBA1 or a control virus encoding GFP (AAV-GFP). As expected, glucocerebrosidase activity was significantly increased (˜7-fold) in the ipsilateral striata of AAV-GBA1-injected mice when compared to the contralateral sides or to AAV-GFP-injected controls (FIG. 4B). Striatal tissue homogenates were also subjected to serial fractionation to separate the cytosolic soluble, membrane-associated and cytosolic insoluble forms of α-synuclein. Quantitation by ELISA revealed that the levels of cytosolic soluble α-synuclein were significantly reduced (86±3% of control, n=5, p<0.01) by striatal expression of glucocerebrosidase (FIG. 4B). The levels of membrane-associated α-synuclein also exhibited a modest reduction (81±9% of control, n=5, p=0.07) upon expression of glucocerebrosidase (FIG. 4B). However, the amount of the insoluble fraction was unchanged by treatment.

The efficacy of glucocerebrosidase in reducing α-synuclein levels in the spinal cords of A53T α-synuclein mice was also determined Newborn A53T α-synuclein mice were injected with AAV-GBA1 or AAV-GFP into both cerebral lateral ventricles and their upper lumbar spinal cords for a total dose of 3E11 drp per pup. As expected, robust expression of glucocerebrosidase (˜3-fold higher than controls) in the spinal cords was achieved following administration of AAV-GBA1 but not the control vector (FIG. 4C). Similar to the striatal injections, administration of AAV-GBA1 lowered α-synuclein levels in the soluble fraction to 67±7% of control (p<0.01, FIG. 4C). Together, these results indicate that augmenting the activity of glucocerebrosidase can lower α-synuclein levels in the CNS of A53T α-synuclein mice.

### Example 6

### Expression of Glucocerebrosidase in A53T α-Synuclein Mouse Brain Decreases Accumulation of Tau Aggregates

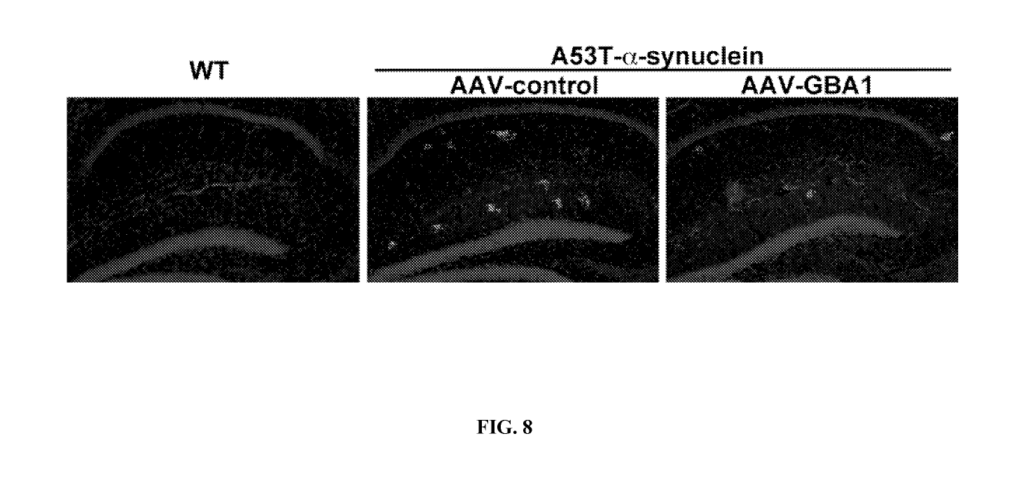

Aggregation of tau has been observed in several animal models including α-synuclein overexpressing mice (Haggerty et al. (2011) Eur J Neurosci 33:1598-1610). To confirm the therapeutic potential of glucocerebrosidase in moderating the accumulation of tau, it was next tested whether this reduction could also be realized in A53T α-synuclein mice. A53T-α-synuclein transgenic mice were injected with either AAV-control or AAV-GBA1 bilaterally at P0. Age-matched, uninjected WT mice were left untreated as negative controls. Analysis of brain tissues of A53T α-synuclein mice showed higher number of aggregates compared to wild-type controls (FIG. 8). Notably, overexpression of GBA1 reduced the number of accumulated tau in age-matched littermates (FIG. 8). The data is consistent with the view that augmenting glucocerebrosidase activity in the CNS can retard the extent of accumulation of pathologically misfolded protein aggregates.

### Example 7

### Expression of Glucocerebrosidase in Tau Transgenic Mice Prevents Memory Dysfunction

Tau transgenic mice (Thy1-TAU22) are a mouse model of Alzheimer's disease and other tauopathies that express human 4-repeat tau mutated at sites G272V and P301S under a Thy1.2-promotor. Thy1-TAU22 displaying tau pathology in the absence of any motor dysfunction and dystonic posture interfering with memory function testing. Thy1-TAU22 shows hyperphosphorylation of tau on several Alzheimer's disease-relevant tau epitopes (AT8, AT100, AT180, AT270, 12E8, tau-pSer396, and AP422), neurofibrillary tangle-like inclusions (Gallyas and MC1-positive) with rare ghost tangles and PHF-like filaments, and mild astrogliosis. These mice also display impaired behavior, including delayed learning and reduced spatial memory.

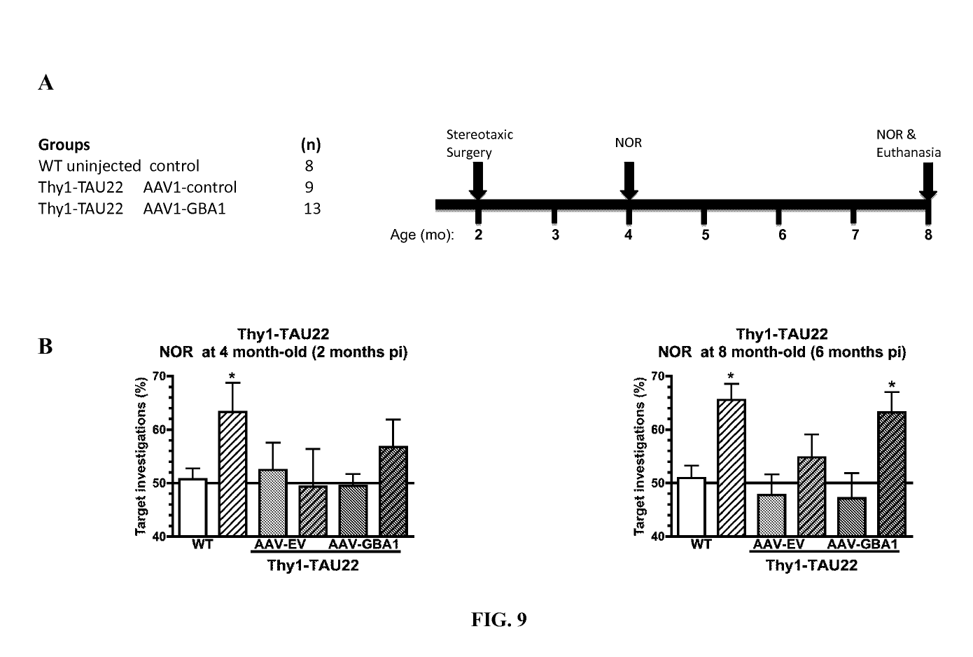

To further evaluate the therapeutic efficacy of augmenting glucocerebrosidase (GBA1) activity, the effects of GBA1 augmentation on tau transgenic mice were assessed. Two month-old Thy1-TAU22 mice were injected with either AAV1-GBA1 or AAV1-control virus (1e13 DRPs/ml). Mice were anesthetized and subjected to stereotaxic injections of the viral vectors into the hippocampus (bilateral hippocampal injections at 3 μl/site (FIG. 9A). Consistent with the Gba1^{D409V/D409V} mice, treatment of Thy1-TAU22 mice with AAV-GBA1 reversed memory deficits (FIG. 9B). There was a trend to cognitive improvement 2 months post-injections that was consolidated when the animals were tested 6 months post-treatment (FIG. 9B). In contrast, Thy1-TAU22 mice treated with the control viral vector showed no preference for the novel object, indicating memory dysfunction at both time points (FIG. 9B). Hence, augmenting GBA1 activity in the CNS of tau transgenic mice corrected memory impairments.

### Discussion

Following the first description of GBA1 mutations as a risk factor for developing PD and DLB, findings from several independent studies have supported a role for glucocerebrosidase in the pathogenesis of these devastating diseases. Both a decrease in glucocerebrosidase activity and the presence of mutant glucocerebrosidase can purportedly induce an increase in CNS levels of α-synuclein/ubiquitin aggregates (see, for example, Xu Y H et al. (2010) Mol Genet Metab 102:436-447; Cullen V et al. (2011) Ann Neurol 69:940-953; Sardi S P et al. (2011) Proc Natl Acad Sci USA 108:12101-12106; Manning-Bog A B et al. (2009) Neurotoxicology 30:1127-1132; and Mazzulli J R et al. (2011) Cell 146:37-52). Analyses of mouse models of Gaucher disease harboring mutations in Gba1 suggest that a decrease in enzymatic activity promotes neuronal protein misprocessing and cognitive deficits, two characteristics of PD and DLB (see, for example, Xu Y H et al. (2010) Mol Genet Metab 102:436-447; Cullen V et al. (2011) Ann Neurol 69:940-953; and Sardi S P et al. (2011) Proc Natl Acad Sci USA 108:12101-1210647). However, the extent to which a deficiency of the enzyme contributes to the pathogenesis of these ailments remains to be determined This study provides further support for a role of glucocerebrosidase in the development of these diseases and validates glucocerebrosidase augmentation in the CNS as a therapeutic approach for diseases associated with α-synuclein misprocessing, such as PD and DLB.

While the precise etiopathologies of PD and LBD remain unclear, the findings of progressive accumulation of α-synuclein and other proteins in LB have implicated protein misfolding as a potential causative mechanism (see, for example, Lee V M et al. (2004) Trends Neurosci 27:129-134 and Dawson T M & Dawson V L (2003) Science 302:819-822). This proteinopathy is replicated in the Gba1^{D409V/D409V} mouse model of Gaucher disease which demonstrate a progressive accumulation of tau pathology in addition to the previously described accumulations of α-synuclein and ubiquitin aggregates. Both α-synuclein and the microtubule-associated protein tau are thought to play pivotal roles in the neurodegenerative processes of several diseases. Mutations in SNCA and MART (the genes encoding for α-synuclein and tau, respectively), with resultant appearances of α-synuclein and tau aggregates, have been implicated in various neurodegenerative diseases, including Alzheimer's disease, PD, DLB and frontotemporal dementia (see, for example, Goris A et al. (2007) Ann Neurol 62:145-153; Lee V M et al. (2004) Trends Neurosci 27:129-134; and Schlossmacher M (2007) α-synuclein and synucleinopathies; and The Dementias 2 ed MN GJR (Butterworth Heinemann, Inc., Oxford), Vol 30, pp 186-215). The mechanisms by which these proteins aggregate appear to be different; for example, α-synuclein can spontaneously self-polymerize (Conway K A et al. (1998) Nat Med 4:1318-1320), while tau requires the presence of an inducing agent (Goedert M et al. (1996) Nature 383:550-553). Moreover, α-synuclein fibrils can reportedly promote the polymerization of tau (Giasson B I et al. (2003) Science 300:636-640 and Waxman E A & Giasson B I (2011) J Neurosci 31:7604-7618). Therefore, it is possible that the observed tau aggregation in the CNS of Gba1^{D409V/D409V} mice occurred secondarily to α-synuclein fibrillization. In addition, only one tau phosphorylated species (Ser202 and Thr205) was increased in aged Gba1^{D409V/D409V} brains. The lack of widespread tau hyperphosphorylation in the Gaucher mouse model suggests that phosphorylation might be a late event, as proposed by Lasagna-Reeves et al. (2012) FASEB J 26:1946-1959.

Although PD typically presents as a movement disorder, it is known to be associated with varying degrees of cognitive impairment, including dementia. PD patients harboring mutations in GBA1 typically have lower cognitive scores than their non-GBA1 mutation-bearing counterparts, suggesting that altered GBA1 increases susceptibility to the development of cognitive deficits (Alcalay R N et al. (2012) Neurology 78:1434-1440). The Gba1^{D409V/D409V} mouse model of Gaucher disease recapitulates many of the aberrant biochemical characteristics noted in brains from PD and DLB patients and the measurable deficits in memory. It has been shown that these disease manifestations can be ameliorated in the CNS of pre-symptomatic animals by supplementation with an exogenous source of the enzyme (Sardi S P et al. (2011) Proc Natl Acad Sci USA 108:12101-12106). Because of the intrinsic difficulties in predicting the development of GBA1-related cognitive impairment, it was pertinent to test whether the same salutary effects can also be realized in animals with overt disease. This example demonstrates that AAV-mediated expression of glucocerebrosidase in both early and late symptomatic Gba1^{D409V/D409V} mice was also effective in reversing cognitive impairment. This recovery in cognition was associated with complete clearance of the glycolipid glucosylsphingosine and measurable reductions in the accumulation of the pathological aggregates. It is possible that augmenting glucocerebrosidase activity in the CNS of Gba1^{D409V/D409V} mice reduced the levels of “toxic” metabolites and thereby improved lysosomal function, which is necessary for correct synaptic function (Hernandez D et al. (2012) Neuron 74:277-284) and proper functioning of pathways that degrade aggregated proteins (Martinez-Vicente M & Cuervo A M (2007) Lancet Neurol 6:352-361 and Cremades N et al. (2012) Cell 149:1048-1059). Importantly, these results strongly suggest that augmenting glucocerebrosidase activity in the CNS may impede the progression of (and even reverse) some of the clinical aspects of Gaucher-related Parkinsonism and associated synucleinopathies.

Ongoing investigations continue to provide greater insights into the relationship between glucocerebrosidase and α-synuclein. It is evident that a decrease in glucocerebrosidase activity or the presence of mutant glucocerebrosidase can promote the aberrant accumulation of α-synuclein (Sardi S P et al. (2012) Neurodegener Dis 10:195-202). Reportedly, α-synuclein can also interact with glucocerebrosidase to reduce its trafficking to the lysosomes or inhibit its activity, thereby exacerbating the disease state (Mazzulli J R et al. (2011) Cell 146:37-52 and Yap T L et al. (2011) J Biol Chem 286:28080-28088). A role for glucocerebrosidase in the disease process is also supported by findings of decreased glucocerebrosidase activity in the brains and CSF of sporadic PD patients, irrespective of whether they harbor GBA1 mutations (Gegg M E et al. (2012) Annals of Neurology 72:455-63). To complement these findings, the above examples describe the study of transgenic A53T α-synuclein mice that overexpress A53T-α-synuclein in the CNS. Measurements of brain lysates from A53T-α-synuclein mice showed that mice with higher levels of α-synuclein were correlated with lower amounts of glucocerebrosidase activity. Importantly, increasing glucocerebrosidase activity in the brains of A53T-α-synuclein mice reduced α-synuclein levels. These results suggest that augmenting glucocerebrosidase activity in the CNS of A53T α-synuclein mice, through its “synuclease” activity, may interrupt the deleterious feedback of α-synuclein on glucocerebrosidase activity and thereby restore the cell's capacity to degrade α-synuclein. Hence, augmenting glucocerebrosidase activity in the CNS via administration of the recombinant enzyme, gene transfer vectors encoding the lysosomal enzyme or small molecule activators of the hydrolase may reduce the extent of accumulation of misfolded proteins and may thereby slow disease progression of PD in subjects with or without GBA1 mutations.

In summary, the efficacy of increasing glucocerebrosidase in modulating the extent of accumulation of aggregates in the CNS was demonstrated in three murine models of tau and α-synuclein proteinopathies. In a symptomatic mouse model of Gaucher-related Parkinsonism and Dementia, augmenting glucocerebrosidase activity in the CNS corrected the aberrant storage of lipids, reversed cognitive dysfunction and reduced the levels of aggregated α-synuclein and tau. Increasing glucocerebrosidase levels in the CNS was also effective in decreasing α-synuclein levels and tau aggregates in the A53T α-synuclein mouse model. Improvement in memory dysfunction was further observed when increasing glucocerebrosidase levels in the CNS of tau transgenic mice. Together, these results support the development of glucocerebrosidase augmentation therapies for PD and related synucleinopathies and tauopathies.

### Materials and Methods

Animals: The Institutional Animal Care and Use Committee at Genzyme, a Sanofi Company, approved all procedures. The Gba1^{D409V/D409V} mouse model of Gaucher disease harbors a point mutation at residue 409 in the murine glucocerebrosidase (Gba1) gene (see, for example, Xu Y H et al. (2003) Am J Pathol 163:2093-2101). Transgenic A53T α-synuclein mice express human A53T α-synuclein (line M83) under the transcriptional control of the murine PrP promoter (Giasson B I et al. (2002) Neuron 34:521-533). Genotyping of A53T α-synuclein mice was performed by quantitative PCR using an Applied Biosystems 7500 real-time PCR system (Life Technologies, Carlsbad Calif.) with the primer-probe set for human SNCA (assay ID Hs00240907_m1). SNCA values were normalized to mouse GADPH (4352339E).

Self-complementary (sc) AAV vectors: The open reading frame of the human GBA1 cDNA was cloned into a shuttle plasmid containing the scAAV2 ITRs and the 0.4 kb GUSB promoter (Passini M A et al. (2010) J Clin Invest (2010) 120:1253-1264). A green fluorescent protein (GFP) open reading frame or a non-coding stater DNA (empty vector, EV) was also cloned into the same shuttle vector. The recombinant plasmids were each packaged into AAV serotype-1 capsids by triple-plasmid transfection of human 293 cells to generate scAAV2/1-GusB-hGBA1 (AAV-GBA1), scAAV2/1-GusB-GFP (AAV-GFP) and scAAV2/1-GusB-EV (AAV-EV). Recombinant AAV vectors were purified by ion-exchange chromatography. The resulting vector preparations of AAV-GBA1, AAV-GFP and AAV-EV typically possessed titers of 1E13 DNAse-resistant particles (drp)/ml.

Stereotaxic injections: Gba1^{D409V/D409V} and A53T α-synuclein mice were anesthetized with isoflurane and subjected to stereotaxic injections of the viral vectors (AAV-GFP, AAV-GBA1, AAV-EV) into the hippocampus (A-P: +2.00; M-L: ±1.50; D-V: −1.5 from bregma and dura; incisor bar: 0.0) or the striatum (A-P: +0.50; M-L: ±2.00; D-V: −2.5 from bregma and dura; incisor bar: 0.0). Two microliters were administered at each injection site using a 10-μl Hamilton syringe (rate of 0.5 μl/min for a total of 2E11 drp/injection site). One hour before surgery and 24 h after surgery, mice were given ketoprofen (5 mg/kg s.c.) for analgesia.

Neonatal injections: On the day of birth (P0), pups received 3 injections (2 μl at each site) into the cerebral lateral ventricles of both hemispheres and the upper lumbar spinal cord. The total dose of AAV-GBA1 and AAV-GFP vectors administered was 3E11 drp per animal. All injections were performed with finely drawn glass micropipette needles as previously described (Passini M A et al. (2010) J Clin Invest 120:1253-1264).

Western blotting: For biochemical analyses, mice were perfused with phosphate-buffered saline (PBS) and processed as previously described (Sardi S P et al. (2012) Neurodegener Dis 10:195-202). Tissues were snap-frozen in liquid nitrogen and stored at −80° C. until assayed. Tissues were homogenized in T-PER lysis buffer (Pierce, Rockford, Ill.) containing a cocktail of protease (Complete®; Roche, Germany) and phosphatase (Pierce, Rockford, Ill.) inhibitors. After centrifugation, lysates were resolved on a 4-12% SDS-PAGE, transferred to nitrocellulose membrane and probed with the following antibodies: mouse anti-tau (Tau-5, 1:500, Millipore, Billerica, Mass.), mouse anti-phosphorylated tau (ATB, Ser202/Thr205; AT180, Thr231; AT270, Thr181; all from Pierce, Rockford, Ill.) or a rabbit anti-β-tubulin antibody (1:1000, Santa Cruz Biotechnology, Santa Cruz, Calif.). The membranes were incubated with infrared secondary (1:10,000) antibodies (LI-COR Biosciences, Lincoln Neb.), and the protein bands visualized by quantitative fluorescence using Odyssey software (LI-COR Biosciences).

Measurements of glucocerebrosidase activity and glycosphingolipid levels: Brain and hippocampal glucocerebrosidase activities were determined as previously described using 4-methylumbelliferyl (4-MU)-β-D-glucoside as the artificial substrate. Hexosaminidase and β-galactosidase activities were determined using 4-MU-N-acetyl-β-D-glucosaminide and 4-MU-β-D-galactopyranoside, respectively. Tissue glucosylceramide (GlcCer) and glucosylsphingosine (GlcSph) levels were measured by mass spectrometry as previously described (Cabrera-Salazar M A et al. Exp Neurol (2010) 225:436-444).

Immunohistochemistry: Tissues were processed as previously described (Sardi S P et al. (2012) Neurodegener Dis 10:195-202). Some tissues were pretreated with proteinase K (1:4 dilution; DAKO, Carpinteria, Calif.) for 7 min at room temperature to expose α-synuclein aggregates. The following primary antibodies were used: mouse anti-ubiquitin (1:50; Millipore, Billerica, Mass.), rabbit anti-α-synuclein (1:300; Sigma, St. Louis, Mo.), and mouse anti-tau (1:500, Tau-5, Millipore, Billerica, Mass.).

Novel object recognition test: The test was conducted as previously described (Sardi S P et al. (2012) Neurodegener Dis 10:195-202). Briefly, mice were individually habituated to explore the open-field box for 5 min on 3 consecutive days. During the first training session (T1), two identical objects were symmetrically placed into the open field 14 inches from each other. Animals were allowed to explore for 5 min. The time spent investigating the objects was recorded using Ethovision video tracking software (Noldus, The Netherlands). After a 24 h retention period, animals were tested (T2) for their recognition of a novel object. Mice were placed back into the open-field box for 5 min, and the time spent investigating the familiar and novel objects was recorded. The results are expressed as percentages of target investigations during training (T1) or testing (T2). A score of 50% investigation on the target represents no preference for either object.

Fractionation and quantification of α-synuclein: Striata and spinal cords from A53T α-synuclein mice were homogenized as previously described (Cullen V et al. (2011) Ann Neurol 69:940-953) to obtain three fractions: cytosolic (Tris-soluble), membrane-associated (Triton-X100-soluble) and insoluble (SDS-soluble). The concentration of human α-synuclein in the different fractions was quantified by sandwich ELISA (Invitrogen, Carlsbad, Calif.). Protein concentration was determined by the microBCA assay (Pierce, Rockford, Ill.).

Statistical analysis: Statistical analyses were performed by Student's t-test or analysis of variance (ANOVA) followed by Newman-Keuls' post-hoc test. Preference for novelty was defined as investigating the novel object more than 50% of the time using a one-sample t-test. All statistical analyses were performed with GraphPad Prism v4.0 (GraphPad Software, San Diego, Calif.). Values of p<0.05 were considered significant.

It is to be understood that while the invention has been described in conjunction with the above embodiments, that the foregoing description and examples are intended to illustrate and not limit the scope of the invention. Other aspects, advantages and modifications within the scope of the invention will be apparent to those skilled in the art to which the invention pertains.

In addition, where features or aspects of the invention are described in terms of Markush groups, those skilled in the art will recognize that the invention is also thereby described in terms of any individual member or subgroup of members of the Markush group.

All publications, patent applications, patents, and other references mentioned herein are expressly incorporated by reference in their entirety, to the same extent as if each were incorporated by reference individually. In case of conflict, the present specification, including definitions, will control.

What is claimed is:

1. A method for improving neural function in a mammal with a proteinopathy comprising administering a therapeutically effective amount of an agent that increases glucocerebrosidase activity in the mammal.

2. The method of claim 1, wherein the mammal has reduced neural function due to the proteinopathy.

3. A method for preventing loss of neural function in a mammal in need thereof comprising administering a therapeutically effective amount of an agent that increases glucocerebrosidase activity.

4. The method of claim 3, wherein the mammal has a proteinopathy.

5. A method for reducing toxic lipids, reducing α-synuclein, reducing tau or inhibiting the accumulation of protein aggregates in a mammal with a proteinopathy comprising administering a therapeutically effective amount of an agent that increases glucocerebrosidase activity.

6. The method of any one of claims 1-5, wherein the mammal has reduced glucocerebrosidase activity prior to administration of the agent.

7. The method of any one of claims 1-6, wherein the mammal has one or more mutations in the glucocerebrosidase 1 (GBA1) gene.

8. The method of claim 7, wherein the mutation is a D409V mutation.

9. The method of claim 5, wherein the method comprises reducing tau.

10. The method of claim 5, wherein the method comprises reducing α-synuclein.

11. The method of claim 5, wherein the method comprises reducing toxic lipids.

12. The method of claim 11, wherein the toxic lipid is glucosylsphingosine.

13. The method of claim 12, wherein the toxic glucosylsphingosine is reduced by at least about 30%.

14. The method of claim 12, wherein the toxic glucosylsphingosine is reduced by at least about 50%.

15. The method of claim 12, wherein the toxic glucosylsphingosine is reduced to a level not significantly different than a mammal without a proteinopathy.

16. The method of claim 5, wherein the method comprises inhibiting the accumulation of protein aggregates.

17. The method of claim 16, wherein the protein aggregates comprise a protein selected from the group consisting of ubiquitin, tau, and α-synuclein.

18. The method of any one of claims 1-17, wherein the mammal has been diagnosed with a disease selected from the group consisting of Alzheimer's disease, Gaucher disease, frontotemporal dementia, progressive supranuclear palsy, Parkinsonism, Parkinson's disease, Lytico-Bodig disease, dementia with Lewy bodies, tangle-predominant dementia, dementia pugilistica, Pick's disease, corticobasal degeneration, Argyrophilic grain disease, ganglioglioma and gangliocytoma, meningioangiomatosis, subacute sclerosing panencephalitis, lead encephalopathy, tuberous sclerosis, Hallervorden-Spatz disease, and lipofuscinosis.

19. The method of any one of claims 1, 2, and 4-18, wherein the proteinopathy comprises protein aggregates.

20. The method of claim 19, wherein the protein aggregates comprise a protein selected from the group consisting of ubiquitin, tau, and α-synuclein.

21. The method of claim 20, wherein the proteinopathy is a tauopathy.

22. The method of claim 21, wherein the tauopathy is a disease selected from the group consisting of Alzheimer's disease, frontotemporal dementia, progressive supranuclear palsy, Parkinsonism, Parkinson's disease, Lytico-Bodig disease, dementia with Lewy bodies, tangle-predominant dementia, dementia pugilistica, Pick's disease, corticobasal degeneration, Argyrophilic grain disease, ganglioglioma and gangliocytoma, meningioangiomatosis, subacute sclerosing panencephalitis, lead encephalopathy, tuberous sclerosis, Hallervorden-Spatz disease, and lipofuscinosis.

23. The method of claim 20, wherein the proteinopathy is a synucleinopathy.

24. The method of any one of claims 1-23, wherein the agent comprises a small molecule, an antibody, a nucleic acid molecule, or a polypeptide.

25. The method of claim 24, wherein the agent is a nucleic acid encoding a GBA1 gene or equivalent thereof.

26. The method of claim 24, wherein the agent is a GBA1 polypeptide or equivalent thereof.

27. The method of claim 24, wherein the agent is an antibody that specifically binds GBA1.

28. The method of claim 24, wherein the agent is a small molecule.

29. The method of claim 28, wherein the small molecule is a small molecule activator of glucocerebrosidase activity.

30. The method of claim 24, wherein the agent is a virus.

31. The method of claim 30, wherein the virus comprises a nucleic acid encoding a GBA1 gene or an equivalent thereof.

32. The method of claim 25 or 31, wherein the GBA1 gene or equivalent thereof is operably linked to a promoter that regulates expression of the GBA1 protein.

33. The method of any one of claims 30-32, wherein the virus infects neuronal cells.

34. The method of any one of claims 30-33, wherein the virus is an adeno-associated virus (AAV).

35. The method of claim 34, wherein the AAV comprises an AAV1, AAV2, AAV3, AAV4, AAV5, AAV6, AAV7, AAV8, AAVrh8, AAV9, AAV10, AAVrh10, AAV11, or AAV12 serotype capsid.

36. The method of claim 34 or 35, wherein the AAV comprises an AAV serotype capsid from Clades A-F.

37. The method of claim 34, wherein the AAV comprises an AAV serotype 1 capsid.

38. The method of any one of claims 34-37, wherein the AAV comprises an AAV1, AAV2, AAV3, AAV4, AAV5, AAV6, AAV7, AAV8, AAVrh8, AAV9, AAV10, AAVrh10, AAV11, or AAV12 inverted terminal repeat (ITR).

39. The method of any one of claims 34-38, wherein the AAV comprises an AAV ITR from Clades A-F.

40. The method of claim 38, wherein the AAV comprises an AAV serotype 2 ITR.

41. The method of any one of claims 34-40, wherein the ITR and the capsid are derived from the same AAV serotype.

42. The method of any one of claims 34-40, wherein the ITR and the capsid are derived from different AAV serotypes.

43. The method of claim 42, wherein the AAV comprises an AAV1 capsid and an AAV2 ITR.

44. The method of any one of claims 34-43, wherein the AAV is a self-complementary AAV.

45. The method of claim 44, wherein the nucleic acid comprises a first heterologous polynucleotide sequence encoding a GBA1 transgene and a second heterologous polynucleotide sequence encoding a complement of the GBA1 transgene, wherein the first heterologous polynucleotide sequence can form intrastrand base pairs with the second polynucleotide sequence.

46. The method of claim 45, wherein the first heterologous polynucleotide sequence and the second heterologous polynucleotide sequence are linked by a mutated AAV ITR.

47. The method of claim 46, wherein the mutated AAV ITR comprises a deletion of the D region and comprises a mutation of the terminal resolution sequence.

48. The method of any one of claims 32-47, wherein the promoter is capable of expressing the GBA1 gene or equivalent thereof in neurons of the central nervous system (CNS).

49. The method of any one of claims 32-48, wherein the promoter comprises a human β-glucuronidase promoter or a cytomegalovirus enhancer linked to a chicken β-actin promoter.

50. The method of any one of claims 1-49, wherein the agent is in a pharmaceutical composition.

51. The method of claim 50, wherein the pharmaceutical composition further comprises a pharmaceutically acceptable carrier.

52. The method of any one of claims 1-51, wherein the agent or pharmaceutical composition is administered by injection.

53. The method of claim 52, wherein the agent or pharmaceutical composition is administered into the CNS.

54. The method of claim 53, wherein the agent or pharmaceutical composition is administered via direct injection into the spinal cord, via intrathecal injection, via intracerebroventricular injection, or via intrahippocampal injection.

55. The method of any one of claims 1-54, wherein the method comprises increasing the glucocerebrosidase activity over baseline levels in a neuron of the mammal.

56. The method of claim 55, wherein the method comprises increasing the glucocerebrosidase activity by at least about 2 fold over baseline levels in the neuron of the mammal.

57. The method of claim 55, wherein the method comprises increasing the glucocerebrosidase activity by at least about 3 fold over baseline levels in the neuron of the mammal.


SEQUENCE LISTING (1 sequence)

SEQ ID NO: 1 - protein, 497 aa, Homo sapiens
ARPCIPKSFGYSSVVCVCNATYCDSFDPPTFPALGTFSRYESTRSGRRMELSMGPIQANHTGTGLLLTLQPEQKFQKVKGFGGAMTDAAALNILALSPPAQNLLLKSYFSEEGIGYNIIRVPMASCDFSIRTYTYADTPDDFQLHNFSLPEEDTKLKIPLIHRALQLAQRPVSLLASPWTSPTWLKTNGAVNGKGSLKGQPGDIYHQTWARYFVKFLDAYAEHKLQFWAVTAENEPSAGLLSGYPFQCLGFTPEHQRDFIARDLGPTLANSTHHNVRLLMLDDQRLLLPHWAKVVLTDPEAAKYVHGIAVHWYLDFLAPAKATLGETHRLFPNTMLFASEACVGSKFWEQSVRLGSWDRGMQYSHSIITNLLYHVVGWTDWNLALNPEGGPNWVRNFVDSPIIVDITKDTFYKQPMFYHLGHFSKFIPEGSQRVGLVASQKNDLDAVALMHPDGSAVVVVLNRSSKDVPLTIKDPAVGFLETISPGYSIHTYLWRRQ